(19) United States
(12) Patent Application Publication
(10) Pub. No.: US 2010/0216837 A1
(43) Pub. Date: Aug. 26, 2010

(54) GLYCINE TRANSPORT INHIBITORS

(75) Inventors: Andrea Bozzoli, Verona (IT); Daniel Marcus Bradley, Essex (GB); Steven Coulton, Essex (GB); Martin Leonard Gilpin, Essex (GB); Jacqueline Anne MacRitchie, Essex (GB); Roderick Alan Porter, Essex (GB); Kevin Michael Thewlis, Essex (GB)

Correspondence Address: GlaxoSmithKline;GLOBAL PATENTS -US, UW2220, P. O. BOX 1539, KING OF PRUSSIA, PA, 19406-0939, US

(73) Assignee: GLAXO GROUP LIMITED, Greenford, Middlesex (GB)

(21) Appl. No.: 11/722,210

(22) PCT Filed: Dec. 21, 2005
(86) PCT No.: PCT/GB2005/004937

§ 371 Date: Jan. 14, 2010

(30) Foreign Application Priority Data
Dec. 23, 2004 (GB) 0428231.5
May 5, 2005 (GB) 0509204.4

(51) Int. Cl. A61K 31/4709; C07C 231/06; A61K 31/166; C07D 215/50; A61K 31/47; C07D 333/06; A61K 31/381; C07D 207/04; A61K 31/40; A61P 25/00

(52) U.S. Cl. 514/314; 564/124; 514/617; 546/169; 514/311; 549/77; 514/438; 548/568; 514/428

(57) ABSTRACT

The present invention relates to compounds of formula (I), or salts or solvates thereof, their use in the manufacture of medicaments for treating neurological and neuropsychiatric disorders, in particular psychoses, dementia or attention deficit disorder. The invention further comprises processes to make these compounds and pharmaceutical formulations thereof.

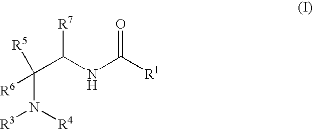

The present invention relates to glycine transporter inhibiting compounds, their use in the manufacture of medicaments for treating neurological and neuropsychiatric disorders, in particular psychoses, dementia or attention deficit disorder. The invention further comprises processes to make these compounds and pharmaceutical formulations thereof.

Molecular cloning has revealed the existence in mammalian brains of two classes of glycine transporters, termed GlyT1 and GlyT2. GlyT1 is found predominantly in the forebrain and its distribution corresponds to that of glutamatergic pathways and NMDA receptors (Smith, et al., Neuron, 8, 1992: 927-935). Molecular cloning has further revealed the existence of three variants of GlyT1, termed GlyT-1a, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995: 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Putffarcken, Science, 262, 689-695 (1993); Lipton and Rosenberg, New Engl. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Glycine transport inhibitors are already known in the art, for example as disclosed in published international patent application WO03/055478 (SmithKline Beecham).

However, there still remains the need to identify further compounds that can inhibit GlyT1 transporters, including those that inhibit GlyT1 transporters selectively over GlyT2 transporters.

It has now been found that a novel class of compounds inhibit GlyT1 transporters and are thus useful in the treatment of certain neurological and neuropsychiatric disorders, including schizophrenia.

Thus, in a first aspect, there is provided a compound of formula (I) or a salt or solvate thereof:

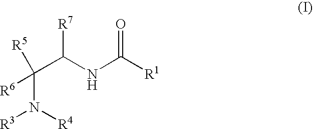

wherein
R⁷ is selected from the group consisting of phenyl substituted with one or more groups R², benzyl optionally substituted with one or more groups R², thiophene optionally substituted with one or more groups R², furan optionally substituted with one or more groups R², thiazole optionally substituted with one or more groups R², oxazole optionally substituted with one or more groups R², pyridyl optionally substituted with one or more groups R², and C₁₋₄alkyl optionally substituted with one or more groups R^{2′};
R² is selected from the group consisting of halogen, cyano, C₁₋₄alkoxy, C₁₋₄alkyl, haloC₁₋₄alkyl, haloC₁₋₄alkoxy, C₃₋₇cycloalkyl, C(O)NR⁹R¹⁰, (where each of R⁹ and R¹⁰ is independently hydrogen or C₁₋₄alkyl, or R⁹ and R¹⁰ together with the nitrogen atom to which they are attached form a 4-, 5-, 6- or 7-membered saturated carbocyclic ring, the 4-, 5-, 6- or 7-membered saturated ring optionally further comprising an additional heteroatom group selected from O, N and S(O)ₘ (where m is 0, 1, or 2)), C₃₋₇cycloalkylC₁₋₄alkoxy, C₁₋₄alkylthio, and haloC₁₋₄alkylthio;
R^{2′} is selected from the group consisting of halogen, cyano, C₁₋₄alkoxy, haloC₁₋₄alkoxy, C₃₋₇cycloalkyl, C(O)NR⁹R¹⁰, (where each of R⁹ and R¹⁰ is independently hydrogen or C₁₋₄alkyl, or R⁹ and R¹⁰ together with the nitrogen atom to which they are attached form a 4-, 5-, 6- or 7-membered saturated carbocyclic ring, the 4-, 5-, 6- or 7-membered saturated ring optionally further comprising an additional heteroatom group selected from O, N and S(O)ₘ (where m is 0, 1, or 2)), C₃₋₇cycloalkylC₁₋₄alkoxy, C₁₋₄alkylthio, and haloC₁₋₄alkylthio;
R³ and R⁴ are independently selected from the group consisting of hydrogen and C₁₋₄alkyl, optionally substituted with one or more groups Y; or R³ and R⁴ together with the nitrogen atom to which they are attached form a saturated or partially unsaturated 4-, 5- 6- or 7-membered carbocyclic ring optionally substituted with a group Y′;
Y is selected from the group consisting of C₁₋₄alkoxy, hydroxy, haloC₁₋₄alkoxy and C₃₋₅cycloalkyl;
Y′ is selected from the group consisting of C₁₋₄alkyl, C₁₋₄alkoxy, halogen, hydroxy, haloC₁₋₄alkoxy, C₃₋₅cycloalkyl and C₅₋₁₀aryl or Y′ forms a —CH₂— or —CH₂—CH₂— bridge between two atoms on the 4-, 5- or 6-membered carbocyclic ring;
R⁵ and R⁸ are independently C₁₋₄alkyl, optionally substituted with one or more groups X; or R⁵ and R⁶ together with the carbon atom to which they are attached form a saturated 5- or 6-membered carbocyclic ring optionally substituted with one or more groups X′, in the case of R⁵ and R⁶ together with the carbon atom to which they are attached forming a 5-membered saturated carbocyclic ring, that ring may optionally further comprising an additional heteroatom group selected from O, N and S(O)ₘ; where m=0, 1 or 2.
X is selected from the group consisting of halogen, hydroxy, C₁₋₄alkoxy, haloC₁₋₄alkyl, haloC₁₋₄alkoxy and C₅₋₁₀aryl;

X′ is selected from the group consisting of halogen, hydroxy, C₁₋₄alkyl, C₁₋₄alkoxy, haloC₁₋₄alkyl, haloC₁₋₄alkoxy and C₅₋₁₀aryl.

and R¹ is selected from a) and b) wherein
a) is a group selected from:

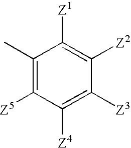

wherein
Z¹ is selected from the group consisting of C₁₋₄alkyl, C₃₋₆cycloalkyl, C₁₋₄alkoxy, C₁₋₄alkylthio, haloC₁₋₄alkyl, phenyl, haloC₁₋₄alkoxy, halophenyl, C₁₋₄alkylsulfoxy, C₁₋₄alkylsulfonyl, bromo and chloro;
Z² is selected from the group consisting of hydrogen, halogen, cyano, C₁₋₄alkyl, phenyl, haloC₁₋₄alkoxy, halophenyl, C₁₋₄alkoxyC₁₋₄alkyl and C₃₋₆cycloalkyl;
Z³ is selected from the group consisting of hydrogen, halogen, C₁₋₄alkyl, C₁₋₄alkoxy, C₁₋₄alkylthio, haloC₁₋₄alkyl, haloC₁₋₄alkoxy, and C₃₋₆cycloalkyl;
Z⁴ is selected from the group consisting of hydrogen, halogen, C₁₋₃alkyl, haloC₁₋₄alkyl, C₁₋₄alkoxy, C₁₋₄alkylthio, phenyl, haloC₁₋₄alkoxy, halophenyl, C₁₋₄alkoxyC₁₋₄alkyl and C₃₋₆cycloalkyl;
Z⁵ is selected from the group consisting of hydrogen, fluoro, chloro, bromo, iodo, hydroxy, C₁₋₄alkyl, C₁₋₄alkoxy, C₁₋₄alkylthio, phenyl, haloC₁₋₄alkyl, haloC₁₋₄alkoxy, halophenyl, C₁₋₄alkoxyC₁₋₄alkyl and C₃₋₆cycloalkyl;
whereby if more than one of Z¹ to Z⁵ is methoxy, then only Z¹ and Z⁵ are methoxy;
b) is a group selected from

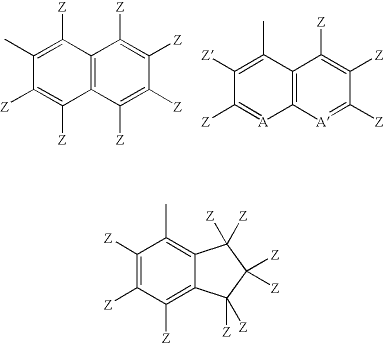

wherein A and A′ are each selected from CZ and N, and A and A′ are not both simultaneously N;
Z′ is selected from: hydrogen, halogen, C₃₋₇cycloalkyl, C₁₋₄alkyl, haloC₁₋₄alkyl and C₁₋₄alkoxyC₁₋₄alkyl,
Each Z is independently selected from hydrogen, halogen, C₃₋₇cycloalkyl, C₁₋₄alkyl, haloC₁₋₄alkyl, C₁₋₄alkoxyC₁₋₄alkyl, C₁₋₄alkoxy, haloC₁₋₄alkoxy, C₁₋₄alkylthio, haloC₁₋₄alkylthio, C₁₋₄alkylsulphoxy, C₁₋₄ alkylsulphonyl, C₁₋₄ dialkylamino, furanyl, piperidinyl and cyano;
and at most 2 groups Z (or where appropriate Z and Z′ together) are not hydrogen.

As used herein, the term “alkyl” refers to a straight or branched alkyl group in all isomeric forms. Examples of C₁₋₄alkyl include methyl, ethyl, propyl, isopropyl, butyl, isobutyl, sec-butyl and tert-butyl.

As used herein, the term “cycloalkyl” refers to a non-aromatic cyclic saturated hydrocarbon ring. Examples of C₃₋₆cycloalkyl include cyclopropyl, cyclobutyl, cyclopentyl, and cyclohexyl.

As used herein, the term “alkoxy” refers to the group —O-alkyl wherein alkyl is as defined above.

As used herein, the term “alkylthio” refers to the group —S-alkyl wherein alkyl is as defined above.

As used herein, the term “alkylsulfoxy” refers to the group —S(O)-alkyl wherein alkyl is as defined above.

As used herein, the term “alkysulfonyl” refers to the group —S(O)₂-alkyl wherein alkyl is as defined above.

As used herein, the term “C₅₋₁₀aryl” refers to a 5- or 6-membered monocyclic aromatic group or a 8- to 10-membered bicyclic aromatic group. Examples of C₅₋₁₀aryl include phenyl, indenyl, azulenyl and naphthyl.

As used herein, the terms “halogen” and its abbreviation “hal” refer to fluorine, chlorine, bromine, or iodine.

As used herein, the term “haloalkyl” refers to an alkyl group as defined above which is substituted with any number of fluorine, chlorine, bromine, or iodine atoms, including with mixtures of those atoms. A haloalkyl group may, for example contain 1, 2 or 3 halogen atoms. For example, a haloalkyl group may have all hydrogen atoms replaced with halogen atoms. Examples of haloalkyl groups include fluoromethyl, difluoromethyl and trifluoromethyl.

As used herein, the term “salt” refers to any salt of a compound according to the present invention prepared from an inorganic or organic acid or base, quaternary ammonium salts and internally formed salts. Physiologically acceptable salts are particularly suitable for medical applications because of their greater aqueous solubility relative to the parent compounds. Such salts must clearly have a physiologically acceptable anion or cation. Suitably physiologically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-physiologically acceptable anion or cation are within the scope of the invention as useful intermediates for the preparation of physiologically acceptable salts and/or for use in non-therapeutic, for example, in vitro, situations.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include' water, ethanol and acetic acid. Most preferably the solvent used is water.

In one embodiment, R⁷ is selected from the group consisting of phenyl substituted with one or more groups R², benzyl optionally substituted with one or more groups R², thiophene optionally substituted with one or more groups R² and C₁₋₄alkyl optionally substituted with one or more groups R².

In one embodiment, R⁷ is selected from the group consisting of phenyl substituted with one or more groups R², unsubstituted benzyl, unsubstituted thiophene and unsubstituted C₁₋₄alkyl.

In one embodiment, R⁷ is phenyl substituted with one or more groups R². For example, there may be 1 or 2 R² groups present in the molecule,

In one embodiment, R² is selected from the group consisting of halogen, C₁₋₄alkoxy, C₁₋₄alkyl, haloC₁₋₄alkyl, and haloC₁₋₄alkoxy. In one embodiment, R² is selected from the group consisting of methoxy, fluoro, chloro, methyl and trifluoromethyl.

In one embodiment, R³ and R⁴ are both simultaneously the same C₁₋₄alkyl, the same C₁₋₄alkyl substituted with one or more groups Y, or R³ and R⁴ together with the nitrogen atom to which they are attached form a saturated 5- or 6-membered carbocyclic ring optionally substituted with a group Y′.

In one embodiment, R³ and R⁴ are both C₁₋₄alkyl, for example methyl or ethyl, for example methyl.

Y may, for example, be selected from the group consisting of C₁₋₄alkoxy, haloC₁₋₄alkoxy and C₅₋₁₀aryl. In one embodiment, Y is selected from the group consisting of C₁₋₄alkoxy and C₅₋₁₀aryl.

Y′ may, for example, be selected from the group consisting of halogen, C₁₋₄alkyl, C₁₋₄alkoxy, haloC₁₋₄alkoxy and C₅₋₁₀aryl. In one embodiment, Y′ is selected from the group consisting of C₁₋₄alkyl, C₁₋₄alkoxy and C₅₋₁₀aryl.

In one embodiment, R³ and R⁴ are independently selected from hydrogen, C₁₋₄alkyl (for example methyl and ethyl), optionally substituted with a group Y, or R³ and R⁴ together with the nitrogen atom to which they are attached form a saturated or partially unsaturated (for example saturated) 4-, 5-, 6- or 7-membered carbocyclic ring optionally substituted with a group Y′.

In a further embodiment, R³ and R⁴ are selected from methyl and ethyl, optionally substituted with a group Y, or R³ and R⁴ together with the nitrogen atom to which they are attached form a saturated 4-, 5- or 6-membered carbocyclic ring optionally substituted with a group Y′. For example, R³ and R⁴ are both unsubstituted methyl, or R³ and R⁴ together with the nitrogen atom to which they are attached form a saturated 5- or 6-membered carbocyclic ring.

In one embodiment, R³ and R⁴ are independently selected from hydrogen and unsubstituted methyl, or R³ and R⁴ together with the nitrogen atom to which they are attached form a saturated 5-membered carbocyclic ring;

Y may, for example, be selected from the group consisting of C₁₋₄alkoxy, hydroxy, C₃₋₅cycloalkyl and C₅₋₁₀ aryl.

Y′ may, for example, be selected from the group consisting of halogen and C₁₋₄alkyl or Y′ may form a —CH₂— bridge between two atoms on the 5- or 6-membered ring. In one embodiment, Y′ is selected from the group consisting of halogen and C₁₋₄alkyl.

In one embodiment, R⁵ and R⁶ are both simultaneously the same C₁₋₄alkyl, the same C₁₋₄alkyl substituted with one or more groups X, or R⁵ and R⁶ together with the carbon atom to which they are attached form a saturated 5- or 6-membered carbocyclic ring optionally substituted with a group X′, the 5- or 6-membered carbocyclic saturated ring optionally further comprising an additional heteroatom group selected from O, N and S(O)ₘ (where m is 0, 1, or 2);

In a further embodiment, R⁵ and R⁶ together with the carbon atom to which they are attached form a saturated 5- or 6-membered carbocyclic ring, for example a 5-membered carbocyclic ring.

X is, for example, selected from the group consisting of halogen, C₁₋ₛalkoxy, haloC₁₋₄alkyl, haloC₁₋₄alkoxy and C₅₋₁₀aryl.

In one embodiment, R⁵ and R⁶ are independently selected from C₁₋₄alkyl (for example methyl and ethyl), optionally substituted with one or more groups X; or R⁵ and R⁶ together with the carbon atom to which they are attached form a saturated 5- or 6-membered carbocyclic ring and in the case of R⁵ and R⁶ together with the carbon atom to which they are attached forming a 5-membered saturated carbocyclic ring, that ring may optionally further comprise an oxygen heteroatom.

For example, in one embodiment R⁵ and R⁶ are independently selected from methyl and ethyl, or R⁵ and R⁶ together with the carbon atom to which they are attached form a saturated 5-membered carbocyclic ring. For example, in a further embodiment, R⁵ and R⁶ are both methyl, or R⁵ and R⁶ together with the carbon atom to which they are attached form a saturated 5-membered carbocyclic ring.

X may, for example, be selected from the group consisting of hydroxy and C₁₋₄alkoxy.

X′ may, for example, be selected from the group consisting of hydroxy and C₁₋₄alkoxy.

In one embodiment, at least one of the pairs of groups R³/R⁴ and R⁵/R⁸ forms a cyclic group with the nitrogen or carbon atom to which they are respectively attached. For example, that cyclic group may be a 5-membered carbocyclic ring.

When R¹ is a), then R¹ is the group

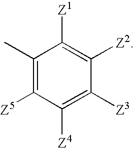

Wherein Z¹ to Z⁵ are as defined in formula (I).

When R¹ is a), then:

In one embodiment of the invention,

Z¹ is selected from the group consisting of chloro, C₁₋₄alkyl, haloC₁₋₄alkyl, C₁₋₄alkoxy, haloC₁₋₄alkoxy, C₁₋₄alkylthio, phenyl, and halophenyl;
Z² is selected from the group consisting of hydrogen, iodo, bromo, chloro, fluoro, C₁₋₄alkyl, haloC₁₋₄alkyl, haloC₁₋₄alkoxy, phenyl, and halophenyl;
Z³ is selected from the group consisting of hydrogen, iodo, bromo, chloro, haloC₁₋₄alkyl, C₁₋₄alkoxy, haloC₁₋₄alkoxy;
Z⁴ is selected from the group consisting of hydrogen, iodo, bromo, chloro, fluoro, C₁₋₄alkoxy, haloC₁₋₄alkoxy, phenyl, and halophenyl; and
Z⁵ is selected from the group consisting of hydrogen, iodo, bromo, chloro, C₁₋₄alkoxy, haloC₁₋₄alkoxy, phenyl, and halophenyl;
wherein no more than three of Z¹, Z², Z³, Z⁴, and Z⁵ are hydrogen.

In another embodiment, Z¹ is selected from the group consisting of chloro, haloC₁₋₄alkyl, C₁₋₄alkoxy, haloC₁₋₄alkoxy, phenyl, and halophenyl, and Z², Z³, Z⁴ and Z⁵ are hydrogen.

In a further embodiment,

Z¹ is selected from the group consisting of chloro, C₁₋₄alkyl, and C₁₋₄alkoxy;
Z² is selected from the group consisting of hydrogen, haloC₁₋₄alkyl, and C₁₋₄alkyl;
Z³ is hydrogen;
Z⁴ is hydrogen; and
Z⁵ is selected from the group consisting of hydrogen, and C₁₋₄alkyl;
wherein no more than three of Z¹, Z², Z³, Z⁴, and Z⁵ are hydrogen.

In one embodiment, Z¹ is selected from the group consisting of C₁₋₄alkyl, C₃₋₈cycloalkyl, C₁₋₂alkoxy, C₁₋₄alkylthio, haloC₁₋₄alkyl, phenyl, haloC₁₋₄alkoxy, halophenyl, C₁₋₄alkylsulfoxy, C₁₋₄alkylsulfonyl, bromo and chloro.

In one embodiment, Z¹ is selected from the group consisting of C₁₋₄alkyl, C₁₋₂alkoxy, C₁₋₄alkylthio, haloC₁₋₄alkyl, phenyl, haloC₁₋₄alkoxy, halophenyl and chloro;

For example, Z¹ is selected from the group consisting of C₁₋₄alkyl, C₁₋₂alkoxy, C₁₋₄alkylthio, haloC₁₋₄alkyl, and chloro, particularly from the group consisting of C₁₋₄alkyl and C₁₋₂alkoxy. For example, Z¹ may be selected from methyl and methoxy.

In one embodiment, Z² is selected from the group consisting of hydrogen, halogen, C₁₋₄alkyl, phenyl and haloC₁₋₄alkyl. For example, Z² may be selected from the group consisting of hydrogen, halogen, and C₁₋₄alkyl. For example Z² may be hydrogen

In one embodiment, Z³ is selected from the group consisting of hydrogen, halogen, C₁₋₄alkyl, C₁₋₄alkoxy and haloC₁₋₄alkyl. For example, Z³ may be selected from the group consisting of hydrogen, halogen C₁₋₄alkyl and haloC₁₋₄alkyl. For example Z³ is hydrogen or trifluoromethyl.

In one embodiment, Z⁴ is selected from the group consisting of hydrogen, halogen, C₁₋₃alkyl, phenyl, C₁₋₄alkoxy and haloC₁₋₄alkyl. For example Z⁴ may be selected from the group consisting of hydrogen and halogen. For example Z⁴ may be hydrogen.

In one embodiment, Z⁵ is selected from the group consisting of hydrogen, hydroxyl, fluoro, chloro, bromo, C₁₋₄alkyl, C₁₋₄alkoxy, haloC₁₋₄alkyl and haloC₁₋₄alkoxy; Z⁵ may be selected from the group consisting of bromo, C₁₋₄alkyl, C₁₋₄alkoxy and haloC₁₋₄alkyl. For example, Z⁵ may be selected from the group consisting of bromo, methyl, methoxy and trifluoromethyl.

When R¹ is b), then R¹ is a group selected from

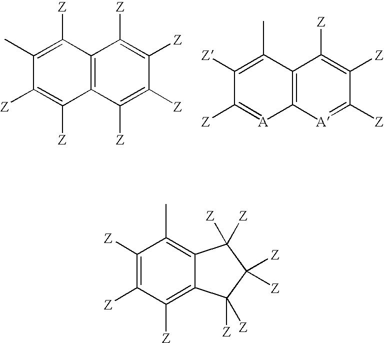

wherein A, A′, Z and Z′ are as defined in formula (I).

When R¹ is b), then,

In one embodiment, R¹ is selected from quinolinyl, naphthyl

In one embodiment, R¹ is selected from groups include quinolinyl, and naphthyl and 2,3-dihydroindenyl, optionally substituted with one or two groups Z or Z′ as appropriate. For example, R¹ is selected from quinolinyl, naphthyl, optionally substituted with one or two groups Z or Z′ as appropriate.

In one embodiment, Z′ is selected from: hydrogen, halogen and C₁₋₄alkyl. For example, Z′ is selected from: hydrogen and methyl.

In one embodiment, Z is selected from the group consisting of hydrogen, halogen, cyano, C₁₋₄alkyl, C₁₋₄alkoxy, haloC₁₋₄alkyl, haloC₁₋₄alkoxy, C₁₋₄alkoxyC₁₋₄alkyl and C₁₋₄cycloalkyl. In a further embodiment, Z is selected from the group consisting of hydrogen, halogen, cyano, C₁₋₄alkyl, C₁₋₄alkoxy, haloC₁₋₄alkyl and haloC₁₋₄alkoxy. In a further embodiment, Z is selected from fluoro, chloro and trifluoromethyl.

In one embodiment, Z is selected from the group consisting of hydrogen, halogen, cyano, C₁₋₄alkyl, C₁₋₄alkoxy, haloC₁₋₄alkyl, haloC₁₋₄alkoxy, C₁₋₄alkoxyC₁₋₄alkyl, C₃₋₆cycloalkyl, C₁₋₄alkylthio, C₁₋₄ dialkylamino, furanyl and piperidinyl. In one embodiment, Z is selected from the group consisting of hydrogen, halogen, cyano, C₁₋₄alkyl, C₁₋₄alkoxy, haloC₁₋₄alkyl, haloC₁₋₄alkoxy, C₁₋₄alkoxyC₁₋₄alkyl, C₃₋₈cycloalkyl, C₁₋₄ dialkylamino, furanyl and piperidinyl.

In a further embodiment, Z is selected from the group consisting of hydrogen, halogen, cyano, C₁₋₄alkoxy, haloC₁₋₄alkyl furanyl, dialkylamino and haloC₁₋₄alkoxy and C₃₋₆cycloalkyl. In a further embodiment, Z is selected from the group consisting of hydrogen, halogen, cyano, methoxy, halomethyl furanyl, dimethylamino and cyclobutyl. In a further embodiment, Z is selected from hydrogen, fluoro, chloro, methoxy and trifluoromethyl.

When R¹ is quinolinyl, Z may for example be hydrogen in all positions other than the 2-position. For example, the remaining groups Z may be hydrogen.

In one embodiment, R¹ is a group selected from a).

Accordingly, in one embodiment, the present invention provides a compound of formula (Ia) or a salt or solvate thereof:

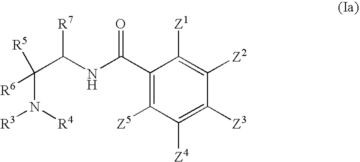

wherein
R⁷ is selected from the group consisting of phenyl substituted with one or more groups R², unsubstituted benzyl, unsubstituted thiophene and unsubstituted C₁₋₄alkyl;
R² is selected from the group consisting of halogen, C₁₋₄alkoxy, C₁₋₄alkyl, haloC₁₋₄alkyl, and haloC₁₋₄alkoxy;
Z¹ is selected from the group consisting of C₁₋₄alkyl, C₁₋₂alkoxy, C₁₋₄alkylthio, and chloro;
Z² is selected from the group consisting of hydrogen, halogen, haloC₁₋₄alkyl, and C₁₋₄alkyl;
Z³ is selected from the group consisting of hydrogen, halogen, haloC₁₋₄alkyl and C₁₋₄alkyl;
Z⁴ is selected from the group consisting of hydrogen and halogen;
Z⁵ is selected from the group consisting of bromo, C₁₋₄alkoxy and haloC₁₋₄alkyl;
R³ and R⁴ are independently selected from hydrogen, C₁₋₄alkyl optionally substituted with a group Y, or R³ and R⁴ together with the nitrogen atom to which they are attached form a saturated or partially unsaturated 4-, 5-, 6- or 7-membered carbocyclic ring optionally substituted with a group Y′;
Y is selected from the group consisting of C₁₋₄alkoxy, hydroxy, C₃₋₅cycloalkyl and C₅₋₁₀ aryl.
Y′ is selected from the group consisting of halogen and C₁₋₄alkyl.

R⁵ and R⁶ are independently selected from C₁₋₄alkyl optionally substituted with one or more groups X; or R⁵ and R⁶ together with the carbon atom to which they are attached form a saturated 5- or 6-membered carbocyclic ring and in the case of R⁵ and R⁶ together with the carbon atom to which they are attached forming a 5-membered saturated carbocyclic ring, that ring may optionally further comprise an oxygen heteroatom; and

X is selected from the group consisting of hydroxy and C₁₋₄alkoxy.

It is to be understood that features of an embodiment of the invention described with reference to one parameter can be combined with the features of another embodiment. The disclosure herein thus includes the combination of the features of any one embodiment with the features of any other embodiment described. All embodiments and features of compounds of formula (I) apply to compounds of formula (Ia).

Examples of compounds of the invention include Examples 1 to 26 shown below, as well as salts and solvates thereof.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms or may exhibit cis-trans isomerism). The individual stereoisomers (enantiomers and diastereoisomers) and mixtures of these are included within the scope of the present invention. Likewise, it is understood that compounds of formula (I) may exist in tautomeric forms other than that shown in the formula and these are also included within the scope of the present invention.

As referred to above, individual enantiomers of compounds of formula (I) may be prepared. In a preferred embodiment, an optically pure enantiomer is desired. The term “optically pure enantiomer” means that the compound contains greater than about 90% of the desired isomer by weight, preferably greater than about 95% of the desired isomer by weight, and most preferably greater than about 99% of the desired isomer by weight, said weight percent based upon the total weight of the isomer(s) of the compound. In some cases, one enantiomer of a particular structure may have a significantly higher activity than the other enantiomer of the same structure. Chirally pure, or chirally enriched compounds may be prepared by chirally selective synthesis or by separation of enantiomers. The separation of enantiomers may be carried out on the final product or, alternatively on a suitable intermediate.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of general formula (I) may be prepared by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognise if a stereocentre exists in compounds of formula (I). Accordingly, the present invention includes both possible stereoisomers and includes not only racemic compounds but the individual enantiomers as well. Where the stereochemistry is indicated as being variable at certain positions, a mixture of stereoisomers may be obtained, this mixture having been separated where indicated. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

Typical reaction routes for the preparation of a compound of formula (I) as hereinbefore defined, are shown in the following schemes. The starting materials and reagents are known to the skilled person in the art and/or can be prepared using methods known in the art.

Compounds of formula (I) can be synthesised by known methods; for example by, but not limited to, the synthetic route outlined in Scheme 1 below

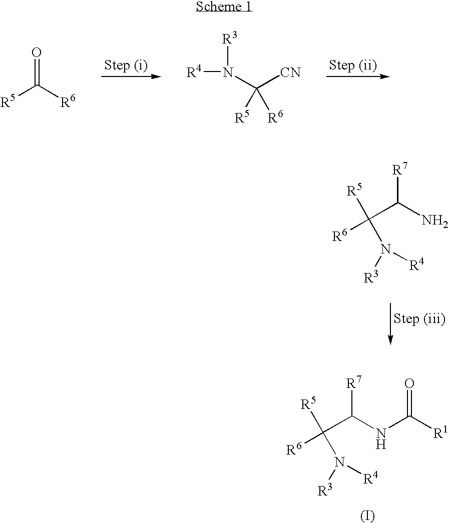

wherein R¹, R³, R⁴, R⁵, R⁶, and R⁷ are as defined for the compound of formula (I).

Step (i) is carried out for example by reaction of a ketone with an amine or amine salt in the presence of inorganic cyanide, for example potassium cyanide, in solvent such as water or by reaction of a ketone with an amine and trimethylsilyl cyanide in either the absence of solvent or in a solvent such as acetic acid.

Step (ii) can be achieved by successive reaction with an appropriate organometallic reagent, in a suitable inert solvent for example tetrahydrofuran, followed by reduction with a reducing agent, for example, sodium borohydride in a suitable solvent, for example methanol.

Acylation step (iii) can be achieved by reaction with a compound of formula (III):

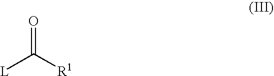

wherein R¹ is as defined in formula (I) and L represents a suitable leaving group. Examples of leaving groups include halogen, hydroxy, OC(═O)alkyl, OC(═O)O-alkyl and OSO₂Me. L may be halogen and acylation in step (iii) may be carried out in an inert solvent such as dichloromethane, in the presence of a base such as triethylamine. When L represents hydroxy, the reaction preferably takes place in an inert solvent such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), polymer-supported EDC, polymer-supported DCC or O-(7-azabenzotriazol-1-yl)-1,1,3,3-tetramethyluronium hexafluoro phosphate (HATU).

Within the scheme there is scope to convert a group R³ into another group R³ and similarly for groups R⁴, R⁵ and R⁶.

Compounds of formula (I) can also be synthesised by the synthetic route outlined in Scheme 2 below. The route is suitable for the synthesis of compounds which have an acid stable group R⁷. For example, it is especially useful for compounds in which R⁷ is phenyl.

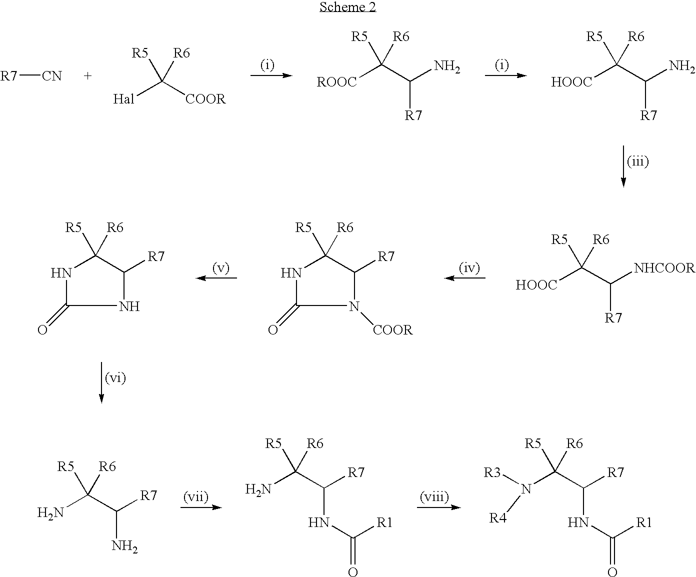

wherein R¹, R³, R⁴, R⁶, R⁶, and R⁷ are as defined for the compound of formula (I), R is on each occasion a suitable alkyl group and L represents a suitable leaving group.

Step (i) is carried out for example by reaction of a nitrile with a suitable compound including a halogen leaving group Hal in the presence of a suitable reducing agent. Examples of leaving groups include bromide. Suitable reducing agents include zinc dust. The reaction may be carried out in a suitable solvent, for example dry THF.

Hydrolysis step (ii) can be achieved by any suitable method, for example by addition of aqueous acid or aqueous alkali.

Amide formation step (iii) may be carried out using suitable amide formation conditions, for example by addition of the appropriate dialkylcarbonate in the presence of aqueous base in a suitable solvent. Suitable solvents include dioxan. Suitable R groups include tertiary butyl.

Cyclisation step (iv) may be carried out by addition of a suitable azide under suitable conditions. For example, diphenylphosphoryl azide may be used in the presence of a suitable base, for example triethylamine.

Step (v) may be carried out by addition of aqueous acid, for example by addition of a mixture of aqueous hydrochloric acid and acetic acid. Steps (v) and (vi) may be carried out in one step by addition of aqueous acid, for example by addition of a mixture of aqueous hydrochloric acid and acetic acid. When R is not tertiary butyl, aqueous alkali may also be used (eg sodium hydroxide).

Acylation step (vii) can be achieved by reaction with a compound of formula (III):

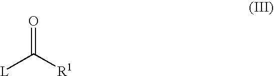

wherein R¹ is as defined in formula (I) and L represents a suitable leaving group. Examples of leaving groups include halogen, hydroxy, OC(═O)alkyl, OC(═O)O-alkyl and OSO₂Me. L may be halogen and acylation in step (iii) may be carried out in an inert solvent such as dichloromethane, in the presence of a base such as triethylamine. When L represents hydroxy, the reaction preferably takes place in an inert solvent such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), polymer-supported EDC, polymer-supported DCC or O-(7-azabenzotriazol-1-yl)-1,1,3,3-tetramethyluronium hexafluoro phosphate (HATU).

N-alkylation step (viii) may be carried out under any suitable N-alkylation conditions. For example, a suitable alkyl compound with a leaving group may be added to the amine in an inert solvent, for example DMF, in the presence of a suitable base, for example triethylamine.

Compounds of the invention may also be prepared by a by the synthetic route outlined in Scheme 3 below. This scheme may be suitable for the synthesis of compounds which have an acid labile group R⁷.

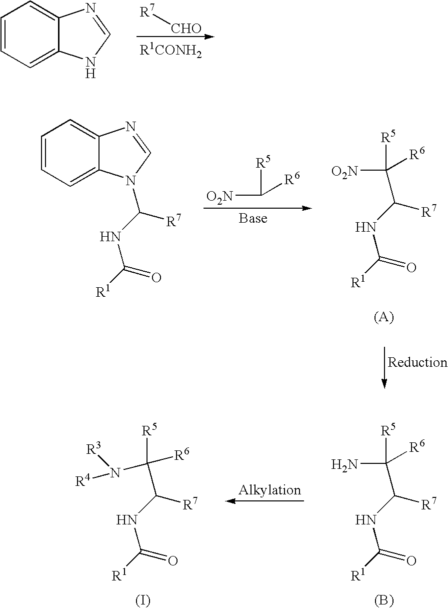

Compounds of type A may be synthesised as described by Katritsky et al (JCS Perkin Trans 1, 1988, 2339 and Gazz. Chim. Italiana 1990, 120, 375). Reduction of the nitro group in A may be achieved under suitable reducing conditions, for example by a catalytic method or by indium metal reduction. Amine B elaborated as in step (viii) in Scheme 2 above.

Accordingly, in a second aspect, the present invention provides a method of preparing a compound of formula (I), comprising the step of:

reacting a compound of formula (II):

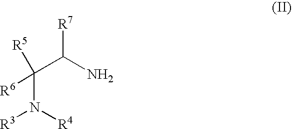

wherein R³, R⁴, R⁵, R⁶ and R⁷ are as defined in formula (I), with a compound of formula (III):

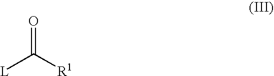

wherein R¹ is as defined in formula (I) and L represents a suitable leaving group;
and thereafter optionally:
removing any protecting groups and/or
converting a compound of formula (I) into another compound of formula (I) and/or
forming a salt or solvate.

Suitable leaving groups L include halogen, hydroxy, OC(═O)alkyl, OC(═O)O-alkyl and OSO₂Me.

Compounds of formula (I) can be converted into further compounds of formula (I) using standard techniques. For example, and by way of illustration rather than limitation, possible conversion reactions include acylation with an appropriate acylating agent such as acetyl chloride, alkylation using an appropriate alkylating reagent such as methyl iodide, and sulfonylation using a sulfonylating agent such as methanesulfonic anhydride and N-alkylation by reductive amination using a ketone or an aldehyde in the presence of a reducing agent such as sodiumtriacetoxy.

Pharmaceutically acceptable salts may be prepared conventionally by reaction with the appropriate acid or acid derivative.

In a further aspect, the present invention provides a compound of formula (II):

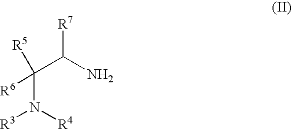

wherein R³, R⁴, R⁵, R⁸ and R⁷ are as defined in formula (I).

Compounds of formula (II) are useful as intermediates in the synthesis of compounds of the invention.

The compounds of the present invention inhibit the GlyT1 transporter. The compounds may selectively inhibit the GlyT1 transporter over the GlyT2 transporter.

Such compounds would be suitable for the treatment of certain neurological and neuropsychiatric disorders. As used herein, the terms “treatment” and “treating” refer to the alleviation and/or cure of established symptoms as well as prophylaxis.

The affinities of the compounds of this invention for the GlyT1 transporter can be determined by the following assay:

HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks were harvested and resuspended at 1.32×10⁶ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds were serially diluted 2.5-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension (1.32×10⁶) and 5 μL of the cell/bead suspension transferred to each well of a 384-well white solid bottom plate (3300 cells/well) containing 100 nL of test compounds. Substrate (5 μL) was added to each well [1:100 dilution of [³H]-glycine stock in assay buffer containing 2.5 μM glycine). Final DMSO concentration was 1% v/v. Data was collected using a Perkin Elmer Viewlux. pIC₅₀ values were determined using ActivityBase.

The following assay may also be used:

HEK293 cells expressing the Glycine (Type 1) transporter are grown in cell medium (DMEM/NUT mix F12) containing 2 mM L-Glutamine, 0.8 mg/mL G418 and 10% heat inactivated fetal calf serum (Gibco BRL) at 37° C. in 5% CO₂. Cells grown to 70-80% confluency in T175 flasks are harvested and resuspended at 4×10⁵ cells/ml in assay buffer [NaCl (140 mM), KCl (5.4 mM), CaCl₂ (1.8 mM), MgSO₄ (0.8 mM), HEPES (20 mM), glucose (5 mM) and alanine (5 mM), pH 7.4]. An equal volume of Leadseeker™ SPA beads (12.5 mg/ml suspended in assay buffer) is added to the cell suspension. Compounds are prepared as 10 mM stocks in DMSO. 2.5 fold serial dilutions of the compounds are made in DMSO from a top conc of 2.5 mM. 100 mL of compound at each concentration is added to the assay plate (384-well white solid bottom plate) using the hummingbird dispenser. 5 uL of the cell/bead mix is then added on top of the compound using a multidrop dispenser. Substrate (5 uL) is then added to each well (1:100 dilution of H3-glycine in assay buffer containing 2.5 uM glycine) Data is collected using a PerkinElmer Viewlux as 5 minute exposures. pIC50 data values are determined using Activity Base.

Compounds may be assayed in their free base form or in the form of a salt, for example the hydrochloride salt or the formate salt. The assays described above are generally considered to provide data that is correct to ±3 standard deviations=±0.5.

Compounds having a pIC₅₀ at the GlyT1 transporter of greater than or equal to 5.0 are considered to be active at the GlyT1 transporter. The example compounds below were found to have a pIC₅₀ at the GlyT1 transporter of greater than or equal to 5.0.

Accordingly, in a further aspect of the invention, there is provided a compound of formula (I) or a salt or solvate thereof: for use in therapy.

In another aspect of the invention, there is provided a compound of formula (I) as hereinbefore described or a salt or solvate thereof, for use in the treatment of a disorder mediated by GlyT1.

As used herein, the term “a disorder mediated by GlyT1” refers to a disorder that may be treated by the administration of a medicament that alters the activity of the GlyT1 transporter. As hereinbefore described, the action of GlyT1 transporters affects the local concentration of glycine around NMDA receptors. As a certain amount of glycine is needed for the efficient functioning of NMDA receptors, any change to that local concentration can affect NMDA-mediated neurotransmission. As hereinbefore described, changes in NMDA-mediated neurotransmission have been implicated in certain neuropsychiatric disorders such as dementia, depression and psychoses, for example schizophrenia, and learning and memory disorders, for example attention deficit disorders and autism. Thus, alterations in the activity of the GlyT1 transporter are expected to influence such disorders.

The disorders mediated by GlyT1 referred to herein include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury.

The compounds of formula (I) are of use as antipsychotic agents for example in the treatment of schizophrenia, schizo-affective disorders, schizophreniform diseases, psychotic depression, mania, acute mania, paranoid and delusional disorders.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of formula (I) are of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of formula (I) are also of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of formula (I) are also of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of formula (I) are also of use in the treatment of substance-related disorders including Substance Use Disorders such as Substance Dependence and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) are also of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of formula (I) are also of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of formula (I) are also of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) are also of use in the treatment of Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).

The compounds of Formula (I) are also of use in the enhancement of cognition including the treatment of cognition impairment in other diseases such as schizophrenia, bipolar disorder, depression, other psychiatric disorders and psychotic conditions associated with cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of formula (I) are also of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The invention also provides a compound of formula (I) as hereinbefore described or a pharmaceutically acceptable salt or solvate thereof for use in the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides a compound of formula (I) as hereinbefore described or a pharmaceutically acceptable salt or solvate thereof for use in the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

In another aspect of the invention, there is provided a method of treating a mammal, including a human, suffering from or susceptible to a disorder mediated by GlyT1, which comprises administering an effective amount of a compound of formula (I) as hereinbefore defined or a salt or solvate thereof.

The invention also provides a method of treating schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders which comprises administering to a mammal in need thereof an effective amount of a compound of formula (I) as hereinbefore described or a pharmaceutically acceptable salt or solvate thereof.

The invention also provides a method of treating psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders which comprises administering to a mammal in need thereof an effective amount of a compound of formula (I) as hereinbefore described or a pharmaceutically acceptable salt or solvate thereof.

The compounds of formula (I) are also of use as anticonvulsants. The compounds of formula (I) are thus useful in the treatment of convulsions in mammals, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures. The invention also provides a method of treating convulsions, which comprises administering to a mammal in need thereof an effective amount of a compound of formula (I) as hereinbefore described or a pharmaceutically acceptable salt or solvate thereof. Treatment of epilepsy may be carried out by the administration of a non-toxic anticonvulsant effective amount of a compound of the formula (III) or a pharmaceutically acceptable salt, or a composition as hereinbefore defined.

The compounds of formula (I) also find use in the treatment of neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

In another aspect of the invention, there is provided use of a compound of formula (I) as hereinbefore defined or a salt or solvate thereof in the preparation of a medicament for the treatment of a disorder mediated by GlyT1.

Preferably, the disorder mediated by GlyT1 to be treated by the use or method as hereinbefore described is a psychosis, including schizophrenia, dementia and attention deficit disorders, particularly schizophrenia.

The invention also provides the use of a compound of formula (I) as hereinbefore described or a pharmaceutically acceptable salt or solvate thereof in the manufacture of a medicament for the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides the use of a compound of formula (I) as hereinbefore described or a pharmaceutically acceptable salt or solvate thereof in the manufacture of a medicament for the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

As used herein, the term “effective amount” means that amount of a drug or pharmaceutical agent that will elicit the biological or medical response of a tissue, system, animal or human that is being sought, for instance, by a researcher or clinician.

Compounds for use according to the invention may be administered as the raw material but the active ingredients are preferably provided in the form of pharmaceutical compositions.

Accordingly, in a further aspect of the invention, there is provided a pharmaceutical composition comprising a compound of formula (I) as hereinbefore described or a salt or solvate thereof, and at least one pharmaceutically acceptable carrier, diluent or excipient.

These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a salt or solvate thereof as a unit dose formulation. If desired, other physiologically active ingredients may also be incorporated in the pharmaceutical compositions of the invention.

It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as atypical antipsychotic drugs and cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divaiproex, carbamazepine and diazepam.

Suitable atypical antipsychotic drugs which may be used in combination of the compounds of the invention include for example risperidone, olanzapine, ziprasidone, aripiprazole and clozapine.

It will be appreciated that the compounds of the combination or composition may be administered simultaneously (either in the same or different pharmaceutical formulations), separately or sequentially.

The compounds of formula (I) and their pharmaceutically acceptable salts and solvates thereof are also suitable for combination with other typical and atypical antipsychotics to provide improved treatment of psychotic disorders. Particular advantages associated with the combinations, uses and methods of treatment of compounds of formula (I) and their pharmaceutically acceptable salts and solvates thereof include equivalent or improved efficacy at doses of administration which are lower than those commonly used for the individual components. Improved treatments of positive symptoms and/or negative symptoms and/or cognitive symptoms of the psychotic disorder may also be observed. The combinations, uses and methods of treatment of the invention may also provide advantages in treatment of patients who fail to respond adequately or who are resistant to treatment with certain neuroleptic agents.

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a pharmaceutically acceptable salt or solvate thereof and at least one neuroleptic agent are within the scope of the current invention. In one embodiment of adjunctive therapeutic administration as described herein, a patient is typically stabilised on a therapeutic administration of one or more of the components for a period of time and then receives administration of another component. Within the scope of this invention, it is preferred that the compounds of formula (I) or a pharmaceutically acceptable salt or solvate thereof is administered as adjunctive therapeutic treatment to patients who are receiving administration of at least one neuroleptic agent, but the scope of the invention also includes the adjunctive therapeutic administration of at least one neuroleptic agent to patients who are receiving administration of compounds of formula (I) or a pharmaceutically acceptable salt or solvate thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a pharmaceutically acceptable salt or solvate thereof to a patient receiving therapeutic administration of at least one neuroleptic agent. In a further aspect, the invention provides the use of compounds of formula (I) or a pharmaceutically acceptable salt or solvate thereof in the manufacture of a medicament for adjunctive therapeutic administration for the treatment of a psychotic disorder in a patient receiving therapeutic administration of at least one neuroleptic agent. The invention further provides compounds of formula (I) or a pharmaceutically acceptable salt or solvate thereof for use for adjunctive therapeutic administration for the treatment of a psychotic disorder in a patient receiving therapeutic administration of at least one neuroleptic agent.

In a further aspect, the invention provides a method of treatment of a psychotic disorder by adjunctive therapeutic administration of at least one neuroleptic agent to a patient receiving therapeutic administration of compounds of formula (I) or a pharmaceutically acceptable salt or solvate thereof. In a further aspect, the invention provides the use of at least one neuroleptic agent in the manufacture of a medicament for adjunctive therapeutic administration for the treatment of a psychotic disorder in a patient receiving therapeutic administration of compounds of formula (I) or a pharmaceutically acceptable salt or solvate thereof. The invention further provides at least one neuroleptic agent for adjunctive therapeutic administration for the treatment of a psychotic disorder in a patient receiving therapeutic administration of compounds of formula (I) or a pharmaceutically acceptable salt or solvate thereof.

In a further aspect, the invention provides a method of treatment of a psychotic disorder by simultaneous therapeutic administration of compounds of formula (I) or a pharmaceutically acceptable salt or solvate thereof in combination with at least one neuroleptic agent. The invention further provides the use of a combination of compounds of formula (I) or a pharmaceutically acceptable salt or solvate thereof and at least one neuroleptic agent in the manufacture of a medicament for simultaneous therapeutic administration in the treatment of a psychotic disorder. The invention further provides the use of compounds of formula (I) or a pharmaceutically acceptable salt thereof in the manufacture of a medicament for simultaneous therapeutic administration with at least one neuroleptic agent in the treatment of a psychotic disorder. The invention further provides compounds of formula (I) or a pharmaceutically acceptable salt thereof for use for simultaneous therapeutic administration with at least one neuroleptic agent in the treatment of a psychotic disorder. The invention further provides the use of at least one neuroleptic agent in the manufacture of a medicament for simultaneous therapeutic administration with compounds of formula (I) or a pharmaceutically acceptable salt thereof in the treatment of a psychotic disorder.

In further aspects, the invention provides a method of treatment of a psychotic disorder by simultaneous therapeutic administration of a pharmaceutical composition comprising compounds of formula (I) or a pharmaceutically acceptable salt or solvate thereof and at least one mood stabilising or antimanic agent, a pharmaceutical composition comprising compounds of formula (I) or a pharmaceutically acceptable salt or solvate thereof and at least one mood stabilising or antimanic agent, the use of a pharmaceutical composition comprising compounds of formula (I) or a pharmaceutically acceptable salt or solvate thereof and at least one mood stabilising or antimanic agent in the manufacture of a medicament for the treatment of a psychotic disorder, and a pharmaceutical composition comprising compounds of formula (I) or a pharmaceutically acceptable salt or solvate thereof and at least one mood stabilising or antimanic agent for use in the treatment of a psychotic disorder.

In a further aspect, the invention provides a kit-of-parts for use in the treatment of a psychotic disorder comprising a first dosage form comprising compounds of formula (I) or a pharmaceutically acceptable salt or solvate thereof and one or more further dosage forms each comprising a neuroleptic agent for simultaneous therapeutic administration.

Within the context of the present invention, the term psychotic disorder includes those disorders mentioned above, such as schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, dyskinetic disorders, depression, bipolar disorder, cognitive impairment and obsessive-compulsive disorders and all the various forms of the disorders as mentioned herein, which are contemplated as part of the present invention.

Examples of neuroleptic/antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; triazine such as lamotrigine; dibenzoxazepines, such as loxapine; dihydroindolones, such as molindone; aripiprazole; and derivatives thereof that have antipsychotic activity.

Examples of neuroleptic drugs that are preferred for use in the present invention are shown in Table A.

TABLE A
Neuroleptic drugs

| | | | | Dosage | |
|---|---|---|---|---|---|
| Common | | Route of | | Range and | |
| Name | Trade Name | Administration | Form | (Median)^{a} | |
| Clozapine | CLOZARIL | oral | tablets | 12.5-900 | mg/day |
| | | | | (300-900 | mg/day) |
| Olanzapine | ZYPREXA | oral | tablets | 5-25 | mg/day |
| | | | | (10-25 | mg/day) |
| Ziprasidone | GEODON | oral | capsules | 20-80 | mg/twice a day |
| | | | | (80-160 | mg/day) |
| Risperidone | RISPERDAL | oral | solution tablets | 2-16 | mg/day tablets |
| | | | | (4-12 | mg/day) |
| Quetiapine | SEROQUEL | oral | tablets | 50-900 | mg/day |
| fumarate | | | | (300-900 | mg/day) |
| Sertindole | SERLECT | | | (4-24 | mg/day) |
| Amisulpride | | | | | |
| Haloperidol | HALDOL | oral | tablets | 1-100 | mg/day |
| | | | | (1-15 | mg/day) |
| Haloperidol | HALDOL | parenteral | injection | | |
| Decanoate | Decanoate | | | | |
| Haloperidol | HALDOL | oral | solution | | |
| lactate | INTENSOL | parenteral | injection | | |
| Chlorpromazine | THORAZINE | rectal | suppositories | 30-800 | mg/day |
| | | oral | capsules | (200-500 | mg/day) |
| | | | solution | | |
| | | | tablets | | |
| | | parenteral | injection | | |
| Fluphenazine | PROLIXIN | | | 0.5-40 | mg/day |
| | | | | (1-5 | mg/day) |
| Fluphenazine | PROLIXIN | parenteral | injection | (about one-half | |
| decanoate | Decanoate | | | the dosage shown | |
| | | | | for oral) | |
| Fluphenazine | PROLIXIN | parenteral | injection | (same as above | |
| enanthate | | | | | |
| Fluphenazine | PROLIXIN | oral | elixer | | |
| hydrochloride | | | solution | | |
| | | parenteral | injection | | |
| Thiothixene | NAVANE | oral | capsules | 6-60 | mg/day |
| | | | | (8-30 | mg/day) |
| Thiothixene | NAVANE | oral | solution | | |
| hydrochloride | | parenteral | injection | | |
| Trifluoperazine | STELAZINE | | | (2-40 | mg/day) |
| Perphenazine | TRILAFON | oral | solution | 12-64 | mg/day |
| | | | tablets | (16-64 | mg/day) |
| | | parenteral | injection | | |
| Perpehazine and | ETRAFON | oral | tablets | | |
| Amitriptyline | TRIAVIL | | | | |
| hydrochloride | | | | | |
| Thioridazine | MELLARIL | oral | suspension | 150-800 | mg/day |
| | | | solution | (100-300 | mg/day) |
| | | | tablets | | |
| Mesoridazine | | | | (30-400 | mg/day) |
| Molindone | MOBAN | | | 50-225 | mg/day |
| | | | | (15-150 | mg/day) |
| Molindone | MOBAN | oral | solution | | |
| hydrochloride | | | | | |
| Loxapine | LOXITANE | | | 20-250 | mg/day |
| | | | | (60-100 | mg/dav) |
| Loxapine | LOXITANE | oral | solution | | |
| hydrochloride | | parenteral | injection | | |
| Loxapine | LOXITANE | oral | capsules | | |
| succinate | | | | | |
| Pimozide | | | | (1-10 | mg/day) |
| Flupenthixol | | | | | |
| Promazine | SPARINE | | | | |
| Triflupromazine | VESPRIN | | | | |
| Chlorprothixene | TARACTAN | | | | |
| Droperidol | INAPSINE | | | | |
| Acetophenazine | TINDAL | | | | |
| Prochlorperazine | COMPAZINE | | | | |
| Methotrimeprazine | NOZINAN | | | | |
| Pipotiazine | PIPOTRIL | | | | |
| Aripiprazole | | | | | |
| Hoperldone | | | | | |

Examples of tradenames and suppliers of selected neuroleptic drugs are as follows: clozapine (available under the tradename CLOZARIL®, from Mylan, Zenith Goldline, UDL, Novartis); olanzapine (available under the tradename ZYPREX®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); thiothixene (available under the tradename NAVANE®; from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman; perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used.

Other preferred neuroleptic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), ziprasidone, and hoperidone.

Particularly preferred neuroleptic agents for use in the invention are olanzapine, risperidone, quetiapine, aripiprazole, haloperidol, clozapine, ziprasidone and osanetant.

It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as atypical antipsychotic drugs and cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

Suitable atypical antipsychotic drugs which which may be used in combination of the compounds of the invention include for example risperidone, olanzapine, ziprasidone, aripiprazole and clozapine.

It will be appreciated that the compounds of the combination or composition may be administered simultaneously (either in the same or different pharmaceutical formulations), separately or sequentially.

For use in medicine, the compounds of the present invention are usually administered as a standard pharmaceutical composition. The present invention therefore provides in a further aspect a pharmaceutical composition comprising a compound of formula (I) as hereinbefore described or a pharmaceutically (i.e. physiologically) acceptable salt thereof and a pharmaceutically (i.e. physiologically) acceptable carrier. The pharmaceutical composition can be for use in the treatment of any of the conditions described herein.

Possible formulations include those suitable for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound, but, where possible, oral administration is preferred.

Formulations suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Formulations suitable for parenteral administration typically comprise sterile aqueous solutions containing a predetermined concentration of the active compound; the solution is preferably isotonic with the blood of the intended recipient. Although such solutions are preferably administered intraveneously, they may also be administered by subcutaneous or intramuscular injection.

Formulations suitable for rectal administration are preferably provided as unit-dose suppositories comprising the active ingredient and one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethylene glycols, alcohols, and combinations thereof.

Compositions suitable for transdermal administration include ointments, gels and patches. Preferably the composition is in unit dose form such as a tablet, capsule or ampoule.

The formulations of the invention may be prepared by any suitable method, typically by uniformly and intimately admixing the active compound(s) with liquids or finely divided solid carriers, or both, In the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

A proposed dose of the active ingredient for use according to the invention for oral, sub-lingual, parenteral, buccal, rectal, intranasal or topical administration to a human (of approximately 70 kg bodyweight) for the treatment of neurological and neuropsychiatric disorders mediated by a GlyT1 inhibitor, including schizophrenia, may be about 1 to about 1000 mg, preferably about 5 to about 500 mg, more preferably about 10 to about 100 mg of the active ingredient per unit dose which could be administered, for example, 1 to 4 times per day.

All publications, including but not limited to patents and patent applications, cited in this specification are herein incorporated by reference as if each individual publication were specifically and individually indicated to be incorporated by reference herein as though fully set forth.

The invention is further illustrated by the following non-limiting examples.

### ABBREVIATIONS

THF tetrahydrofuran
DCM dichloromethane
DMF dimethylformamide
HATU O-(7-azabenzotriazol-1-yl) N,N,N′,N′-tetramethyluroniumhexa fluorophosphate
EDC N-(3-(dimethylamino)propyl)-N-ethylcarbodiimide hydrochloride
HOAt 3H-(1,2,3)-triazolo(4,5-b)pyridine-3-ol
NMP N-methylpyrrolidinone
DIPEA N,N-diisopropylethylamine
HOBt 1-hydroxybenzotriazole hydrate 0.88 ammonia concentrated aqueous ammonia specific gravity 0.88
   Analytical LC/MS chromatography conditions:
Column: Waters Atlantis 50 mm×4.6 mm, 3 um particle size
Mobile phase: A: 0.05% Formic acid+Water
   B: Acetonitrile+0.05% Formic acid
Gradient: 5-min runtime: 3% B to 97% B over 4 min
Flow rate: 3 ml/min
UV wavelength range: 220-330 nm
Temperature: 30° C.

### Mass Directed Auto-Purification System Chromatography Conditions:

Column: Waters Atlantis 19 mm×100 mm or 30 mm×100 mm, Sum particle size
Mobile phase: A: 0.1% Formic acid+Water
   B: Acetonitrile+0.1% Formic acid
Gradient: 13.5 min runtime with 10 min gradient dependant on analytical retention time
Flow rate: 20 or 40 ml/min

### Description 1:1-(Dimethylamino)cyclopentanecarbonitrile

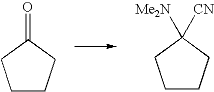

To a suspension of dimethylamine hydrochloride (8.15 g; 0.1 mol) in cyclopentanone (8.4 g; 0.1 mol) at 0° C. was added dropwise a solution of potassium cyanide (6.5 g; 0.1 mol) in water (50 ml) over 10 min. After vigorous stirring at room temperature for 18 h, the crude reaction mixture was extracted three times with diethyl ether (200 ml) and the combined extracts washed twice with water (50 ml), dried over sodium sulphate, filtered and the filtrate evaporated to afford the title product as a pale yellow oil (12.5 g) which was used without further purification. ¹H NMR (CDCl₃) δ: 1.7-2.0 (6H, m), 2.15-2.3 (2H, m), and 3.3 (6H, s). Mass Spectrum (Electrospray LC/MS): Found 112 (MH⁺—HCN). C₈H₁₄N₂ requires 138.

### Description 2: {1-[amino(2-thienyl)methyl]cyclopentyl}dimethylamine

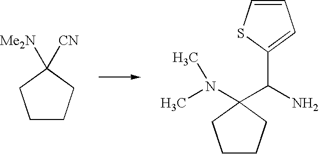

To a stirred, cooled)(−78°, solution of 1-(Dimethylamino)cyclopentanecarbonitrile (D1) (200 mg, 1.45 mmol) in dry THF (5 ml) under argon was added a solution of 2-thienyl lithium (1.6 ml of a 1M solution in THF, 1.60 mmol). This was allowed to warm to room temperature slowly over 3 hours. The reaction mixture was then cooled to 0°, treated with methanol (5 ml) and sodium borohydride (165 mg, 4.35 mmol) and stirred for 18 hours at room temperature. The reaction mixture was then treated with a saturated solution of sodium hydrogen carbonate and evaporated under reduced pressure. The residue was extracted 3 times with DCM, dried over magnesium sulphate and evaporated. Chromatography on silica gel, eluting with 0-80% ethyl acetate in pentane gave a product which was treated with further methanol (5 ml) and sodium borohydride (165 mg, 4.35 mmol) and stirred for 18 hours at room temperature. The reaction mixture was then treated with a saturated solution of sodium hydrogen carbonate and evaporated under reduced pressure. The residue was extracted 3 times with DCM, dried over magnesium sulphate and evaporated. Chromatography on silica gel eluting with DCM followed by DCM/methanol/0.88 ammonia (90:9:1) gave {1-[amino(2-thienyl)methyl]cyclopentyl}dimethylamine as a colourless oil (101 mg, 31%). δ_{H} (400 MHz, CDCl₃) 0.75 (1H, m), 1.3-2.1 (7H, overlapping m), 2.26 (6H, s), 4.61 (1H, s), 6.95 (1H, m), 7.02 (1H, m), 7.19 (1H, m) ppm. LC/MS: m/z (ES+) 208 (MH⁺—NH3, C₁₂H₂ₒN₂S requires 224), Retention time 0.85 minutes.

### Description 3: [1-(1-aminopropyl)cyclopentyl]dimethylamine

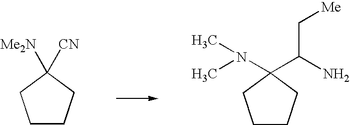

To a stirred, cooled)(−78°, solution of 1-(Dimethylamino)cyclopentanecarbonitrile (D1) (200 mg, 1.45 mmol) in dry THF (5 ml) under argon was added a solution of ethyl lithium (3.2 ml of a 0.5M solution in benzene, 1.60 mmol). This was allowed to warm to room temperature slowly over 3 hours. The reaction mixture was then cooled to 0°, treated with methanol (5 ml) and sodium borohydride (165 mg, 4.35 mmol) and stirred for 18 hours at room temperature. The reaction mixture was then treated with a saturated solution of sodium hydrogen carbonate and evaporated under reduced pressure. The residue was extracted 3 times with DCM, dried over magnesium sulphate and evaporated. Chromatography on silica gel eluting with DCM followed by DCM/methanol/0.88 ammonia (90:9:1) gave [1-(1-aminopropyl)cyclopentyl]dimethylamine as a colourless oil (113 mg, 46%). δ_{H} (400 MHz, CDCl₃) 1.00 (3H, m), 1.25-1.6 (10H, overlapping m), 2.17 (6H, s), 2.87 (1H, m) ppm.

### Description 4: [1-(1-aminopentyl)cyclopentyl]dimethylamine

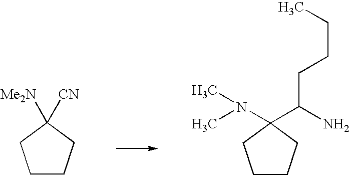

A solution of dry toluene (200 ul, 1.9 mmol) in dry THF (2 ml) was cooled to −78° and treated with n-butyl lithium (1.0 ml of a 1.6M solution in hexane, 1.60 mmol). The cooling bath was removed and the reaction allowed to warm to room temperature and stirred for a further minutes. Recooled to −78° and treated with solution of 1-(dimethylamino)cyclopentanecarbonitrile (D1) (200 mg, 1.45 mmol) in dry THF (2 ml). Allowed to warm to room temperature over the next 3 hours and then treated with methanol (5 ml) and sodium borohydride (165 mg, 4.35 mmol) and stirred for 18 hours at room temperature. The reaction mixture was then treated with a saturated solution of sodium hydrogen carbonate and evaporated under reduced pressure. The residue was extracted 3 times with DCM, dried over magnesium sulphate and evaporated. Chromatography on silica gel eluting with DCM followed by DCM/methanol/0.88 ammonia (90:9:1) gave [1-(1-aminopentyl)cyclopentyl]dimethylamine as a colourless oil (163 mg, 49%). δ_{H} (400 MHz, CDCl₃) 0.90 (3H, m), 1.1-1.7 (14H, overlapping m), 2.16 (6H, s), 2.95 (1H, m) ppm.

### Description 5: [1-(1-amino-2-phenylethyl)cyclopentyl]dimethylamine

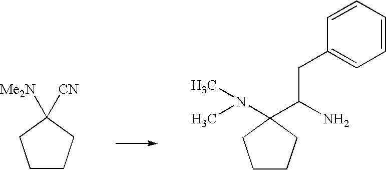

A solution of N,N,N′,N′-tetramethylethylenediamine (200 ul) in toluene (2 ml) under argon was cooled to 0° and treated with n-butyl lithium (1.0 ml of a 1.6M solution in hexane, 1.6 mmol). Stirred at 0° for 1 hour and then cooled to −78° and treated with a solution of 1-(Dimethylamino)cyclopentanecarbonitrile (D1) (200 mg, 1.45 mmol) in dry THF (2 ml). Allowed to warm to room temperature over the next 18 hours and then treated with dry methanol (4 ml) and sodium borohydride (165 mg, 4.35 mmol) and stirred at room temperature for 5 hours. The reaction mixture was then treated with a saturated solution of sodium hydrogen carbonate and evaporated under reduced pressure. The residue was extracted 3 times with DCM, dried over magnesium sulphate and evaporated. Chromatography on silica gel eluting with DCM followed by DCM/methanol/0.88 ammonia (90:9:1) gave [1-(1-amino-2-phenylethyl)cyclopentyl]dimethylamine (28 mg, 8%). δ_{H} (400 MHz, CDCl₃) 1.5-1.75 (8H, overlapping m), 2.23 (6H, s), 2.27 (1H, dd, J=10.4 and 2.4 Hz), 2.93 (1H, dd, J=13.2 and 2.0 Hz), 3.27 (1H, dd, J=10.4 and 2.4 Hz), 7.26 (5H, overlapping m) ppm. LC/MS: m/z (ES+) 233 (MH⁺, C₁₅H₂₄N₂ requires 232), Retention time 1.16 minutes.

### Description 6: [1-(1-amino-2-methylpropyl)cyclopentyl]dimethylamine

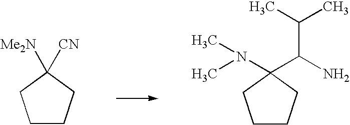

A solution of 1-(dimethylamino)cyclopentanecarbonitrile (D1) (200 mg, 1.45 mmol) in dry THF (4 ml) was cooled to −78° under argon and treated with isopropyl lithium (2.28 ml of a 0.7M solution in pentane, 1.6 mmol). The reaction mixture was allowed to warm to room temperature slowly over the next 3 hours. Water (4 drops) was then added and the mixture evaporated under reduced pressure. The residue was extracted with DCM, dried and evaporated to afford the intermediate which was redissolved in dry THF (5 ml), stirred under argon and treated with lithium borohydride (1.4 ml of a 2M solution in THF, 2.80 mmol). Stirred at 60° for 1 hour and then overnight at room temperature and treated with a saturated solution of sodium hydrogen carbonate and evaporated under reduced pressure. The residue was extracted 3 times with DCM, dried over magnesium sulphate and evaporated. Chromatography on silica gel eluting with DCM followed by DCM/methanol/0.88 ammonia (90:9:1) gave [1-(1-amino-2-methylpropyl)cyclopentyl]dimethylamine as an oil (18 mg, 7%). δ_{H} (400 MHz, CDCl₃) 0.86 (3H, s), 0.95 (3H, s), 1.47-1.75 (8H, overlapping m), 2.12 (1H, m), 2.19 (6H, s), 2.87 (1H, d, J=2.0 Hz) ppm.

### Description 7: ethyl 3-amino-3-(2-fluorophenyl)-2,2-dimethylpropanoate

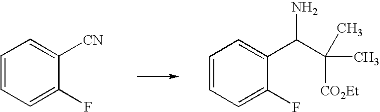

Zinc dust (894 mg, 13.7 mmol) was suspended in dry THF (3 ml) under argon and ethyl bromoisobutyrate (2 drops) added. Heated to reflux for 5 minutes and a solution of 2-fluorobenzonitrile (332 mg, 2.74 mmol) in dry THF (6 ml) was added and reflux maintained for a further 5 minutes. A solution of ethyl bromoisobutyrate (2.13 g, 10.94 mmol) in dry THF (2 ml) was now added dropwise over 1 hour at reflux and under argon. After a further 15 minutes at reflux the mixture was cooled and the solvent removed under reduced pressure. The crude mixture was then resuspended in dry methanol (10 ml) and cooled) (0° during the portionwise addition of sodium borohydride (200 mg, 5.4 mmol). Stirred for 1.5 hours and the solvent removed under reduced pressure. The residue was partitioned between ethyl acetate and saturated sodium hydrogen carbonate solution and the organic layer dried, filtered and evaporated to afford a material which was chromatographed on silica gel. Elution with 0-80% ethyl acetate in pentane gave ethyl 3-amino-3-(2-fluorophenyl)-2,2-dimethylpropanoate as a colourless oil (530 mg, 81%). δ_{H} (400 MHz, CDCl₃) 1.13 (3H, s), 1.14 (3H, s), 1.27 (3H, t, J=7.2 Hz), 4.17 (2H, q, J=7.2 Hz), 4.59 (1H, s), 7.00 (1H, m), 7.12 (1H, m), 7.21 (1H, m), 7.37 (1H, m) ppm. LC/MS: m/z (ES+) 240 (MH⁺, C₁₃H₁₈NO₂F requires 239), Retention time 1.61 minutes.

### Description 8: 3-({[(1,1-dimethylethyl)oxy]carbonyl}amino)-3-(2-fluorophenyl)-2,2-dimethylpropanoic acid

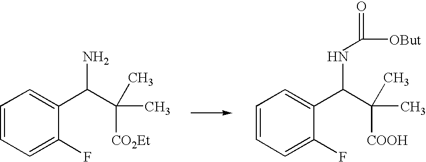

A solution of ethyl 3-amino-3-(2-fluorophenyl)-2,2-dimethylpropanoate (D7) (964 mg, 4.03 mmol) in 50% hydrochloric acid (10 ml) was heated at 100° overnight and the hydrochloric acid then removed under reduced pressure. The resulting solid was redissolved in methanol and the solvent removed under reduced pressure. Finally, the solid was suspended in chloroform and the solvent removed under reduced pressure to afford crude 3-amino-3-(2-fluorophenyl)-2,2-dimethylpropanoic acid as a hydrochloride salt (1.01 g).

A mixture of this salt (0.78 g, 3.15 mmol), dioxan (20 ml), water (2 ml), 2M sodium hydroxide solution (3.30 ml, 6.62 mmol) and di-t-butyl dicarbonate (755 mg, 3.37 mmol) was stirred at room temperature overnight. The dioxan was then removed under reduced pressure and the aqueous residue washed with ether. The ether solution was extracted with saturated sodium hydrogen carbonate solution (3 ml) and the aqueous layer combined with the aqueous reaction mixture. The pH of the solution was adjusted to approximately 4.0 and extracted twice with ethyl acetate. The organic layer was dried and evaporated to afford 3-({[(1,1-dimethylethyl)oxy]carbonyl}amino)-3-(2-fluorophenyl)-2,2-dimethylpropanoic acid as a crisp foam (830 mg, 85%): LC/MS: m/z (ES+) 312 (MH⁺, C₁₆H₂₂NO₄F requires 311), Retention time 2.76 minutes.

### Description 9: 1,1-dimethylethyl 5-(2-fluorophenyl)-4,4-dimethyl-2-oxo-1-imidazolidinecarboxylate

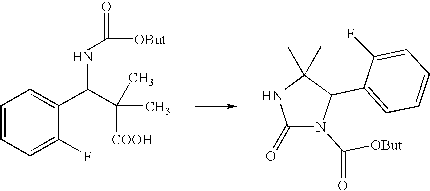

A solution of 3-({[(1,1-dimethylethyl)oxy]carbonyl}amino)-3-(2-fluorophenyl)-2,2-dimethylpropanoic acid (D8) (790 mg, 2.54 mmol) in dry DMF (7 ml) was cooled)(0° under argon and treated with triethylamine (427 ul, 3.05 mmol) and diphenylphosphoryl azide (657 ul, 3.05 mmol). Stirred for 1 hour at 25° and 5 hours at 65°. The reaction mixture was cooled and the solvent removed under reduced pressure. The crude product was dissolved in ethyl acetate and washed with saturated sodium hydrogen carbonate solution. The dried solution was evaporated under reduced pressure to give an oil which was chromatographed on silica gel. Elution with 20-100% ethyl acetate in pentane gave 1,1-dimethylethyl 5-(2-fluorophenyl)-4,4-dimethyl-2-oxo-1-imidazolidinecarboxylate as a colourless gum (706 mg, 90%). δ_{H} (400 MHz, CDCl₃) 0.89 (3H, s), 1.26 (9H, s), 1.52 (3H, s), 4.91 (1H, br s), 5.23 (1H, s), 7.08 (1H, m), 7.16 (1H, m), 7.27 (2H, overlapping m) ppm. LC/MS: m/z (ES+) 331 (MNa⁺, C₁₈H₂₁N₁₂O₃F requires 308), Retention time 2.70 minutes.

### Description 10: 1-(2-fluorophenyl)-2-methyl-1,2-propanediamine dihydrochloride

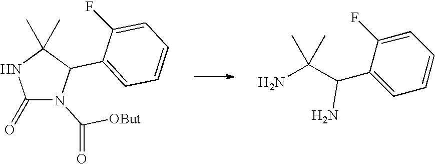

A solution of 1,1-dimethylethyl 5-(2-fluorophenyl)-4,4-dimethyl-2-oxo-1-imidazolidinecarboxylate (D9) (325 mg, 1.05 mmol) in 50% hydrochloric acid (5 ml) and acetic acid (5 ml) was heated at 125° for 4 days. The reaction mixture was cooled and the volatile components evaporated under reduced pressure. The residue was re-evaporated twice from methanol followed by twice from chloroform. The resulting solid was triturated 3 times with chloroform and dried to leave 1-(2-fluorophenyl)-2-methyl-1,2-propanediamine dihydrochloride (211 mg, 78%). LC/MS: m/z (ES+) 183 (MH⁴, C₁₀H₁₅N₂F requires 182), Retention time 0.29 minutes.

### EXAMPLE 1

### (±)-N-{[1-(dimethylamino)cyclopentyl][4-(methyloxy)phenyl]methyl}-2,6-dimethylbenzamide

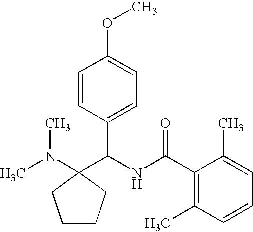

Under an atmosphere of argon, a stirring solution of 4-bromoanisole (187 mg; 1.00 mmol) in anhydrous THF (3 mL) was cooled to −78° C. and tent-butyllithium (1.7M in pentane; 1.2 mL; 2.04 mmol) was added dropwise over 5 minutes. After stirring at this temperature for 30 minutes, a solution of 1-(dimethylamino)cyclopentanecarbonitrile (D1) (138 mg; 1.00 mmol) in anhydrous THF (3 mL) was added dropwise over 5 minutes and the reaction warmed to 20° C. over 3 hours. The mixture was quenched by the sequential addition of methanol (1 mL) and water (3 mL), and the product isolated by partition between ethyl acetate and a saturated aqueous solution of ammonium chloride. The organic phase was dried (Na₂SO₄) and concentrated in vacuo, giving a colourless oil (190 mg). This was dissolved in methanol (10 mL) and sodium borohydride (88 mg; 2.32 mmol) was added portionwise to the stirring mixture. After stirring at 20° C. for 1 hour, the reaction was partitioned between ethyl acetate and a saturated aqueous solution of sodium hydrogen carbonate. The separated organic phase was concentrated in vacuo, dissolved in dichloromethane and purified by SCX column chromatography, giving a yellow oil (108 mg). Half of this (54 mg) was dissolved in dichloromethane (2 mL) and diisopropylethylamine (100 uL; 0.59 mmol) followed by 2,6-dimethylbenzoyl chloride (37 mg; 0.22 mmol) was added. The mixture was shaken for 1 hour and purified by SCX column chromatography, giving the title compound as an off-white solid (62 mg; 0.16 mmol). δ_{H} (400 MHz, CDCl₃) 7.36 (2H, d, J 9), 7.15 (1H, dd, J 8, 8), 7.01 (2H, d, J 8), 6.87 (2H, d, J 9), 5.21 (1H, d, J 6), 3.81 (3H, s), 2.31 (6H, s), 2.28 (6H, s), 1.88-1.24 and 1.02-0.95 (8H, m); LC/MS: m/z (ES+) 381 (MW, C₂₄H₃₂N₂O₂ requires 380), Retention time 1.96 minutes.

### EXAMPLE 2

### (±)-N-[[1-(dimethylamino)cyclopentyl](4-fluorophenyl)methyl]-2-methyoxy-6-(methyl)benzamide

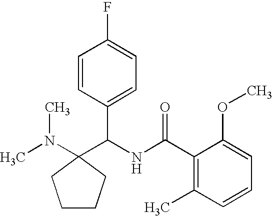

Under an inert atmosphere of argon, tert-butyllithium (2.20 mL; 1.7 M in pentane; 3.74 mmol) was added dropwise over 10 minutes to a cold (−78° C.) stirring solution of 4-fluoriodobenzene (472 mg; 2.13 mmol) in anhydrous diethyl ether. After stirring at this temperature for 40 minutes, 1-(dimethylamino)cyclopentanecarbonitrile (D1) (207 mg; 1.50 mmol) in anhydrous diethyl ether (2 mL) was added dropwise over 3 minutes to the cold (−78° C.) stirring mixture. After stirring at this temperature for 2 hours 50 minutes, the reaction was removed from the cooling bath for 4 minutes, quenched with methanol (5 mL) and warmed to room temperature. Purification by SCX column chromatography gave 214 mg of product, which was dissolved in methanol (5 mL). Sodium borohydride (70 mg; 1.85 mmol) was added in one portion to the stirring solution at room temperature, and the reaction was stirred for 15 hours. Purification by SCX column chromatography gave 1-[amino(4-fluorophenyOmethyl]-N,N-dimethylcyclopentanamine.

HATU (41 mg; 0.11 mmol) was added in one portion to a stirring mixture of 1-[amino(4-fluorophenyl)methyl]-N,N-dimethylcyclopentanamine (20 mg; 71.0 umol), diisopropylethylamine (50 ml; 0.29 mmol), and 2-methyloxy-6-methylbenzoic acid (19 mg; 0.11 mmol) in DMF (1 mL). Upon product formation, the mixture was purified by successive SCX column chromatography and MDAP, giving the title compound as the formate salt (12.2 mg; 31.7 umol). δ_{H} (400 MHz, CDCl₃) 9.99 (1H, br d, J 9), 8.08 (1H, br s), 7.57-7.50 (2H, m), 7.22-7.15 (1H, m), 7.13-7.06 (2H, m), 6.74 (1H, d, J 8), 6.68 (1H, d, J 8), 5.57 (1H, d, J 9), 3.73 (3H, s), 2.86 (6H, s), 2.53-2.40 (1H, m), 2.15 (3H, m), 2.11-1.95 (3H, m), 1.58-1.43 (2H, m), 1.13-1.00 (1H, m), 1.00-0.86 (1H, m); LC/MS: m/z (ES+) 385 (MW, C₂₃H₂₉FN₂O₂ requires 384), retention time 1.96 minutes.

### EXAMPLE 3

### (±)-N-[[1-(dimethylamino)cyclopentyl](4-fluorophenyl)methyl]-2,6-dimethylbenzamide

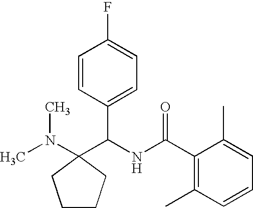

2,6-Dimethylbenzoyl chloride (135 uL; 0.64 mmol) was added in one portion to a stirring solution of 1-[amino(4-fluorophenyl)methyl]-N,N-dimethylcyclopentanamine (75 mg; 0.32 mmol) and diisopropylethylamine (150 uL; 0.88 mmol) in dichloromethane (1.5 mL). After stirring for 2 hours 20 minutes, the reaction was purified by SCX column chromatography giving the title compound as a yellow oil (70 mg, 0.19 mmol). LC/MS: m/z (ES+) 369 (MH⁺, C₂₃H₂₉FN₂O requires 368), retention time 1.96 minutes.

The compounds in the table below were prepared using similar methods to those described for the Examples above. Method: A=Acid chloride; Method H=HATU mediated coupling. Work-up and purification was carried out using appropriate methods similar to those described in the examples above.

TABLE 1

| | | | Mass spectrum | |
|---|---|---|---|---|
| | | | (Electrospray | |
| | | Coupling | LC/MS), API⁺ | |
| Ex | Structure | method | Ret.time (min) | Name |
| 4 | 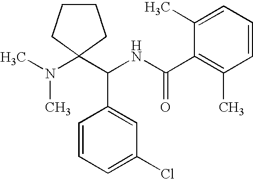 | A | Found 385(MH⁺) C₂₃H₂₉³⁵ClN₂O Requires 384; 2.01 | (±)-N-{(3-chlorophenyl)[1- (dimethylamino)cyclo- pentyl]methyl}-2,6- dimethylbenzamide |
| 5 | 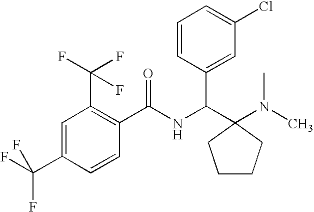 | A | Found 493(MH⁺) C₂₃H₂₃³⁵ClF₆N₂O Requires 492; 2.20 | (±)-N-{(3-chlorophenyl)[1- (dimethylamino)cyclo- pentyl]methyl}-2,4- bis(trifluoromethyl)benzamide |
| 6 | 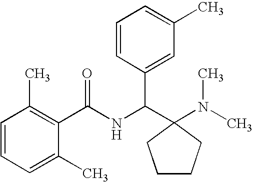 | A | Found 365(MH⁺) C₂₄H₃₂N₂O Requires 364; 1.91 | (±)-N-[[1- (dimethylamino)cyclopentyl](3- methylphenyl)methyl]-2,6- dimethylbenzamide |
| 7 | 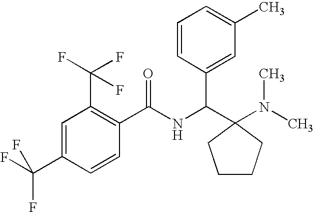 | A | Found 473(MH⁺) C₂₄H₂₆F₆N₂O Requires 472; 2.24 | (±)-N-[[1- (dimethylamino)cyclopentyl](3- methylphenyl)methyl]-2,4- bis(trifluoromethyl)benzamide |
| 8 | 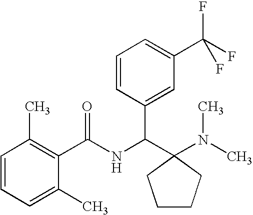 | A | Found 419(MH⁺) C₂₄H₂₉F₃N₂O Requires 418; 2.09 | (±)-N-{[1- (dimethylamino)cyclopentyl][3- (trifluoromethyl)phenyl]methyl}- 2,6-dimethylbenzamide |
| 9 | 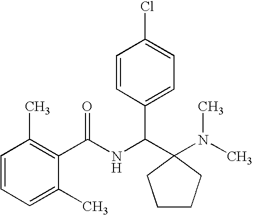 | A | Found 385(MH⁺) C₂₃H₂₉³⁵ClN₂O Requires 384; 2.01 | (±)-N-{(4-chlorophenyl)[1- (dimethylamino)cyclopentyl] methyl}-2,6- dimethylbenzamide |
| 10 | 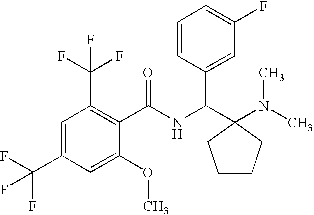 | H | Found 507(MH⁺) C₂₄H₂₅F₇N₂O₂ Requires 506; 2.31 | (±)-N-[[1- (dimethylamino)cyclopentyl] (3-fluorophenyl)methyl]- 2-(methyloxy)-4,6- bis(trifluoromethyl)benzamide |
| 11 | 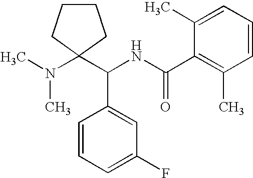 | A | Found 369(MH⁺) C₂₃H₂₉FN₂O Requires 368; 1.93 | (±)-N-[[1- (dimethylamino)cyclopentyl] (3-fluorophenyl)methyl]- 2,6-dimethylbenzamide |
| 12 | 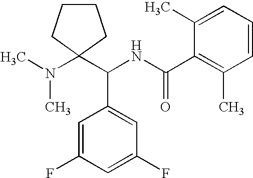 | A | Found 387(MH⁺) C₂₃H₂₈F₂N₂O Requires 286; 1.83 | (±)-N-{(3,5- difluorophenyl)[1- (dimethylamino)cyclopentyl] methyl}-2,6- dimethylbenzamide |
| 13 | 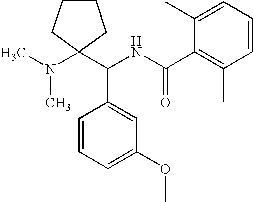 | A | Found 381(MH⁺) C₂₄H₃₂N₂O₂ Requires 380; 1.96 | N-{[1- (dimethylamino)cyclo- pentyl][3- (methyloxy)phenyl]methyl}- 2,6-dimethylbenzamide |
| 14 | 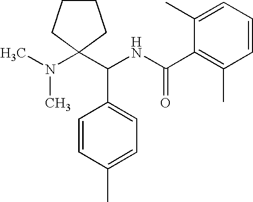 | A | Found 365(MH⁺) C₂₄H₃₂N₂O Requires 364; 2.11 | N-[[1- (dimethylamino)cyclo- pentyl](4- methylphenyl)methyl]-2,6- dimethylbenzamide |

Coupling method: A=Acid chloride (as in Example 3); H=HATU (as in Example 2)

### EXAMPLE 15

### N-[[1-(dimethylamino)cyclopentyl](2-thienyl)methyl]-2-(trifluoromethyl)-4-quinolinecarboxamide

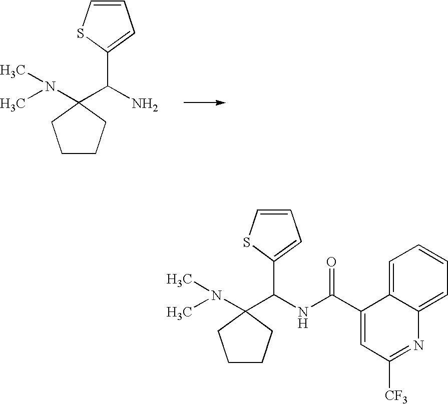

A solution of {1-[amino(2-thienyl)methyl]cyclopentyl}dimethylamine (D2) (100 mg, 0.45 mmol) in dry DCM (3 ml), containing DMF (0.3 ml), was treated with 2-trifluoromethyl-4-quinolinecarboxylic acid (108 mg, 0.45 mmol), EDC (95 mg, 0.50 mmol) and HOBt (31 mg, 0.18 mmol) and left overnight. Washed reaction mixture with saturated sodium hydrogen carbonate and water and dried. Loaded on to a silica gel column and eluted with 0-80% ethyl acetate in pentane to afford Ni-[[1-(dimethylamino)cyclopentyl](2-thienyl)methyl]-2-(trifluoromethyl)-4-quinolinecarboxamide (185 mg, 92%). δ_{H} (400 MHz, CDCl₃) 1.32 (1H, m), 1.43 (1H, m), 1.6 (3H, overlapping m), 1.82 (1H, m), 1.95 (2H, m), 2.29 (6H, s), 5.66 (1H, d, J=6.8 Hz), 7.01 (1H, m), 7.10 (1H, m), 7.15 (1H, m), 7.27 (1H, m), 7.73 (1H, m), 7.79 (1H, s), 7.87 (1H, m), 8.30 (2H, overlapping m) ppm. LC/MS: m/z (ES+) 448 (MH⁺, C₂₃H₂₄N₃OSF₃ requires 447), Retention time 2.11 minutes.

### EXAMPLE 16

### 2-chloro-N-{1-[1-(dimethylamino)cyclopentyl]propyl}-3-(trifluoromethyl)benzamide

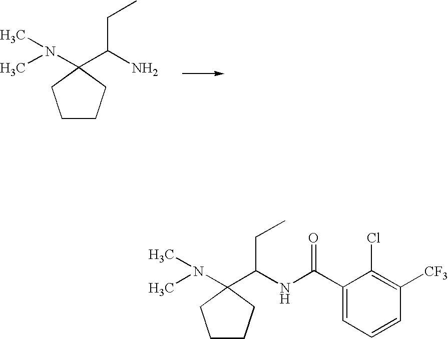

A solution of [1-(1-aminopropyl)cyclopentyl]dimethylamine (D3) (113 mg, 0.66 mmol) and 2-chloro-3-trifluoromethylbenzoyl chloride (161 mg, 0.66 mmol) in dry DCM (2 ml) was treated with triethylamine (185 ul, 1.32 mmol) and stirred overnight at room temperature. The reaction mixture was washed with saturated sodium hydrogen carbonate, dried over magnesium sulphate and applied to a silica gel column. Elution with 20-100% ethyl acetate in pentane gave 2-chloro-N-{1-[1-(dimethylamino)cyclopentyl]propyl}-3-(trifluoromethyl)benzamide as a white solid (202 mg, 81%). δ_{H} (400 MHz, CDCl₃) 1.10 (3H, m), 1.3-2.0 (10H, overlapping m), 2.26 (6H, s), 4.36 (1H, m), 5.86 (1H, d, J=9.2 Hz), 7.43 (1H, dd, J=8.0 and 8.0 Hz), 7.65 (1H, d, J=8.0 Hz), 7.76 (1H, d, J=8.0 Hz) ppm. LC/MS: m/z (ES+) 377 (MH⁺, C₁₈H₂₄N₂O³⁵ClF₃requires 376), Retention time 1.95 minutes.

### EXAMPLE 17

### N-{1-[1-(dimethylamino)cyclopentyl]propyl}-2-(trifluoromethyl)-4-quinolinecarboxamide

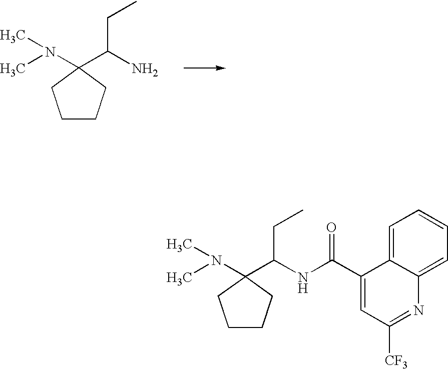

Prepared from D3 as described in Example 15. δ_{H} (400 MHz, CDCl₃) 1.13 (3H, m), 1.3-2.0 (1011, overlapping m), 2.30 (6H, s), 4.48 (1H, m), 6.01 (1H, d, J=9.6 Hz), 7.75 (1H, s), 7.75 (1H, dd, J=8.0 and 8.0 Hz), 7.86 (1H, dd, J=8.0 and 8.0 Hz), 8.26 (1H, d, J=8.0 Hz), 8.37 (1H, d, J=8.0 Hz) ppm. LC/MS: m/z (ES+) 394 (MH⁺, C₂₁H₂₆N₃OF₃ requires 393), Retention time 1.99 minutes.

### EXAMPLE 18

### N-{1-[1-(dimethylamino)cyclopentyl]pentyl}-2-(trifluoromethyl)-4-quinolinecarboxamide

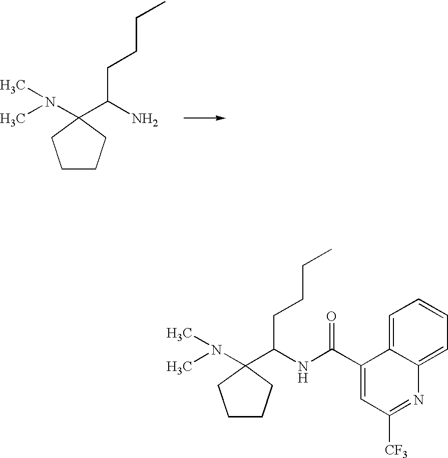

Prepared from D4 as described in Example 15. δ_{H} (400 MHz, CDCl₃) 0.95 (3H, m), 1.35-1.85 (14H, overlapping m), 2.30 (6H, s), 4.56 (1H, m), 6.02 (1H, d, J=9.6 Hz), 7.73 (1H, s), 7.73 (1H, dd, J=8.0 and 8.0 Hz), 7.86 (1H, dd, J=8.0 and 8.0 Hz), 8.26 (1H, d, J=8.0 Hz), 8.38 (1H, d, J=8.0 Hz) ppm. LC/MS: m/z (ES+) 422 (MH⁺, C₂₃H₃₀N₃OF₃ requires 421), Retention time 2.24 minutes.

### EXAMPLE 19

### N-{1-[1-(dimethylamino)cyclopentyl]-2-phenylethyl}-2-(trifluoromethyl)-4-quinolinecarboxamide

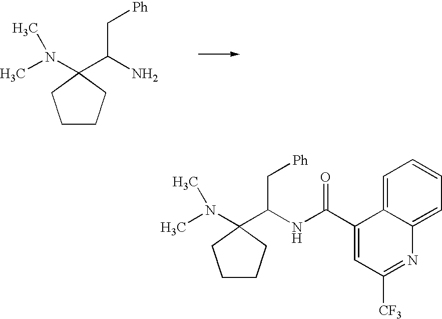

Prepared from D5 as described in Example 15. δ_{H} (400 MHz, CDCl₃) 1.5-2.0 (8H, overlapping m), 2.40 (6H, s), 2.49 (1H, dd, J=14.4 and 11.6 Hz), 3.50 (1H, dd, J=14.4 and 4.0 Hz), 5.07 (1H, m), 5.80 (1H, d, J=9.2 Hz), 7.3 (6H, overlapping m), 7.52 (2H, overlapping m), 7.78 (1H, dd, J=8.0 and 8.0 Hz), 8.17 (1H, d, J=8.0 Hz) ppm. LC/MS: m/z (ES+) 456 (MH⁺, C₂₆H₂₈N₃OF₃ requires 455), Retention time 2.21 minutes.

### EXAMPLE 20

### N-{1-[1-(dimethylamino)cyclopentyl]-2-methylpropyl}-2-(trifluoromethyl)-4-quinolinecarboxamide

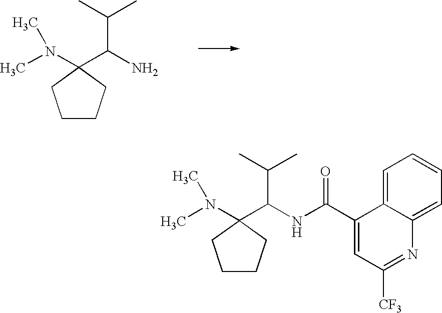

Prepared from D6 as described in Example 15. δ_{N} (400 MHz, CDCl₃) 0.98 (3H, d), 1.18 (3H, d), 1.4-1.9 (8H, overlapping m), 2.28 (6H, s), 2.34 (1H, m), 4.52 (1H, dd, J=9.6 and 2.8 Hz), 6.17 (1H, d, J=10.0 Hz), 7.72 (1H, s), 7.74 (1H, dd, J=8.0 and 8.0 Hz), 7.88 (1H, dd, J=8.0 and 8.0 Hz), 8.29 (2H, dd, J=8.0 and 8.0 Hz) ppm. LC/MS: m/z (ES+) 408 (MH⁺, C₂₂H₂₈N₃OF₃ requires 407), Retention time 2.10 minutes.

### EXAMPLE 21

### N-[[1-(dimethylamino)cyclopentyl](2-thienyl)methyl]-2,3-dimethylbenzamide

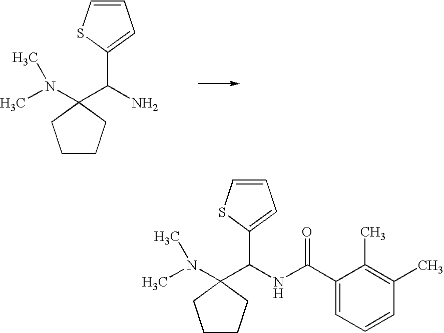

Prepared from D2 as described in Example 15. δ_{N} (400 MHz, CDCl₃) 1.4-1.9 (8H, overlapping m), 2.27 (6H, s), 2.30 (3H, s), 2.32 (3H, s), 5.64 (1H, d, J=7.6 Hz), 6.60 (1H, d, J=7.6 Hz), 6.95 (1H, m), 7.05 (1H, m), 7.14 (1H, m), 7.2 (3H, overlapping m) ppm. LC/MS: m/z (ES+) 357 (MH⁺, C₂₁H₂₈N₂OS requires 356), Retention time 1.95 minutes.

### EXAMPLE 22

### 2-chloro-N-[[1-(dimethylamino)cyclopentyl](2-thienyl)methyl]-3-(trifluoromethyl)benzamide

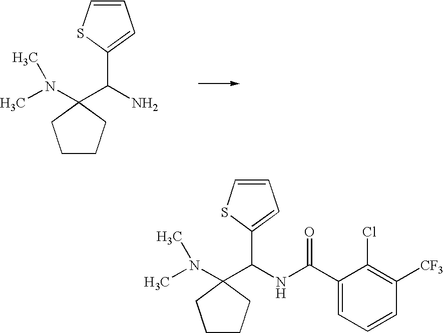

Prepared from D2 as described in Example 15. δ_{H} (400 MHz, CDCl₃) 1.4-1.9 (8H, overlapping m), 2.27 (6H, s), 5.57 (1H, d, J=6.8 Hz), 6.97 (1H, m), 7.07 (2H, overlapping m), 7.21 (1H, m), 7.46 (1H, dd, J=7.6 and 7.6 Hz), 7.70 (1H, dd, J=7.6 and 1 Hz), 7.77 (1H, dd, J=7.6 and 1 Hz) ppm. LC/MS: m/z (ES+) 431 (MH⁺, C₂₀H₂₂N₂O³⁵ClF₃S requires 430), Retention time 2.13 minutes.

### EXAMPLE 23

### N-[[1-(dimethylamino)cyclopentyl](2-thienyl)methyl]-2,6-dimethylbenzamide

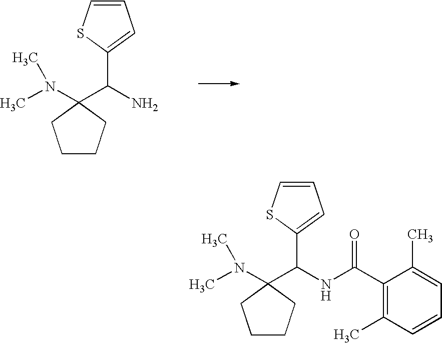

Prepared from D2 as described in Example 16. δ_{H} (400 MHz, CDCl₃) 1.23 (1H, m), 1.5-1.9 (7H, overlapping m), 2.25 (6H, s), 2.32 (6H, s), 5.62 (1H, d, J=7.2 Hz), 6.46 (1H, d, J=7.2 Hz), 6.96 (1H, m), 7.02 (2H, m), 7.08 (1H, m), 7.16 (1H, m), 7.20 (1H, m) ppm. LC/MS: m/z (ES+) 357 (MH⁺, C₂₁H₂₈N₂OS requires 356), Retention time 1.85 minutes.

### EXAMPLE 24

### N-[2-amino-1-(2-fluorophenyl)-2-methylpropyl]-2,6-dimethylbenzamide

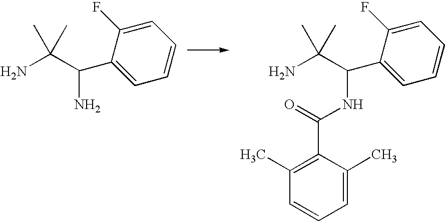

A solution of 1-(2-fluorophenyl)-2-methyl-1,2-propanediamine dihydrochloride (D10) (207 mg, 0.81 mmol), triethylamine (560 ul, 4.0 mmol) and 2,6-dimethylbenzoyl chloride (136 mg, 0.81 mmol) in dry DCM (10 ml) was stirred at room temperature overnight. The reaction mixture was washed with saturated sodium hydrogen carbonate solution, dried and evaporated to give crude product which was chromatographed on silica gel. Elution with 20-100% ethyl acetate in pentane gave N-[2-amino-1-(2-fluorophenyl)-2-methylpropyl]-2,6-dimethylbenzamide as a white solid (155 mg, 61%). δ_{H} (400 MHz, CDCl₃) 1.10 (3H, s), 1.32 (3H, s), 2.24 (6H, s), 5.34 (1H, d, J=8.8 Hz), 7.00 (2H, m), 7.1-7.4 (8H, overlapping m) ppm. LC/MS: m/z (ES+) 315 (MH⁺, C₁₉H₂₃N₂OF requires 314), Retention time 1.64 minutes.

### EXAMPLE 25

### N-[1-(2-fluorophenyl)-2-methyl-2-(1-pyrrolidinyl)propyl]-2,6-dimethylbenzamide

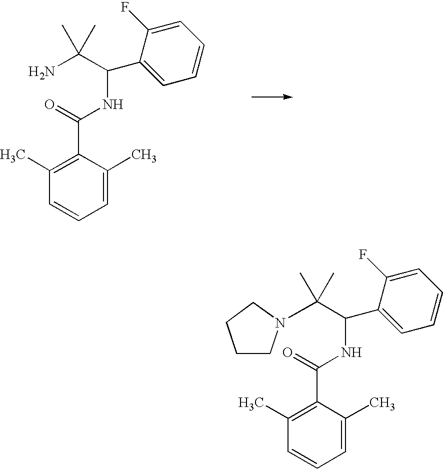

A suspension of N-[2-amino-1-(2-fluorophenyl)-2-methylpropyl]-2,6-dimethylbenzamide (E24) (93 mg, 0.296 mmol), 1,4-dibromobutane (432 mg, 2.0 mmol) and potassium carbonate (552 mg, 4.0 mmol) in dry DMF (2 ml) was heated at 50° for 3 hours and then stirred at room temperature overnight. Heated at 50° for a further 4 hours after which time reaction was complete, as judged by LCMS. The solvent was removed under reduced pressure and the residue partitioned between water (2 ml) and ethyl acetate. The organic layer was dried over magnesium sulphate and evaporated to afford the crude product. This was chromatographed on silica gel, eluting with 0-80% ethyl acetate in pentane to give a product which still contained a less polar impurity. Purification by Mass Directed Auto-Purification gave N-[1-(2-fluorophenyl)-2-methyl-2-(1-pyrrolidinyl)propyl]-2,6-dimethylbenzamide formate salt as a white solid (61 mg, 56%). δ_{H} (400 MHz, CDCl₃) 1.20 (3H, s), 1.49 (3H, s), 1.96-2.13 (4H, overlapping m), 2.21 (6H, s), 3.22 (4H, broad s), 5.80 (1H, d, J=8.4 Hz), 6.96 (2H, d, J=7.6 Hz), 7.12 (2H, overlapping m), 7.20 (1H, dd, J=7.6 and 7.6 Hz), 7.30 (1H, m), 7.51 (1H, m) 8.09 (1H, s) 10.10 (1H, broad s) ppm. LC/MS: m/z (ES+) 369 (MH⁺, C₂₃H₂₉N₂OF requires 368), Retention time 1.84 minutes.

### EXAMPLE 26

### N-[1-(2-fluorophenyl)-2-methyl-2-(1-pyrrolidinyl)propyl]-2-(methyloxy)-4,6-bis(trifluoromethyl)benzamide

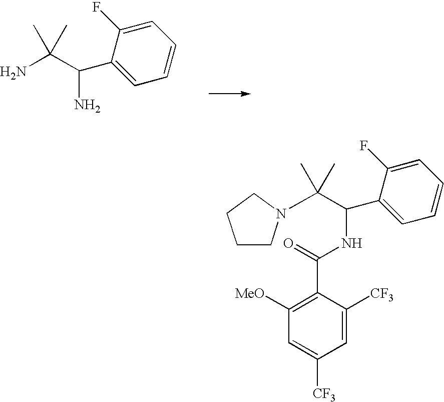

Prepared as described in Examples 25 and 26 as a formate salt. δ_{H} (400 MHz, CDCl₃) 1.25 (3H, s), 1.55 (3H, s), 2.07 (2H, m), 2.17 (2H, m), 3.34 (2H, broad m), 3.44 (2H, broad m), 3.85 (3H, s), 5.83 (1H, d, J=8.4 Hz), 7.07 (1H, m), 7.21 (1H, m), 7.25 (1H, s obscurred), 7.32 (1H, m), 7.47 (1H, s), 7.50 (1H, m), 8.10 (1H, s), 11.5 (1H, broad s) ppm. LC/MS: m/z (ES+) 507 (MH⁺, C₂₄H₂₅N₂O₂F₇ requires 506), Retention time 2.30 minutes.

The compounds of the Examples above were convertable to their corresponding hydrochloride salts by dissolving the parent free base in DCM or DCM/methanol mixtures and adding 1M hydrogen chloride in ether, followed by evaporation and drying in vacuo. Compounds purified by Mass Directed Auto-Purification were isolated as the formate salt which could be converted to the free base via an SCX column and to the corresponding hydrochloride salt by reaction with 1M hydrogen chloride in ether as described above.

1. A compound of compound of formula (I) or a salt or solvate thereof: 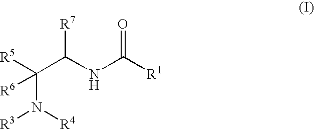
wherein
R⁷ is selected from the group consisting of phenyl substituted with one or more groups R², benzyl optionally substituted with one or more groups R², thiophene optionally substituted with one or more groups R², furan optionally substituted with one or more groups R², thiazole optionally substituted with one or more groups R², oxazole optionally substituted with one or more groups R², pyridyl optionally substituted with one or more groups R², and C₁₋₄alkyl optionally substituted with one or more groups R^{2′};
R² is selected from the group consisting of halogen, cyano, C₁₋₄alkoxy, C₁₋₄alkyl, haloC₁₋₄alkyl, haloC₁₋₄alkoxy, C₃₋₇cycloalkyl, C(O)NR⁹R¹⁰, (where each of R⁹ and R¹⁹ is independently hydrogen or C₁₋₄alkyl, or R⁹ and R¹⁹ together with the nitrogen atom to which they are attached form a 4-, 5-, 6- or 7-membered saturated carbocyclic ring, the 4-, 5-, 6- or 7-membered saturated ring optionally further comprising an additional heteroatom group selected from O, N and S(O)ₘ (where m is 0, 1, or 2)), C₃₋₇cycloalkylC₁₋₄alkoxy, C₁₋₄alkylthio, and haloC₁₋₄alkylthio;
R^{2′} is selected from the group consisting of halogen, cyano, C₁₋₄alkoxy, haloC₁₋₄alkoxy, C₃₋₇cycloalkyl, C(O)NR⁹R¹⁰, (where each of R⁹ and R¹⁹ is independently hydrogen or C₁₋₄alkyl, or R⁹ and R¹⁹ together with the nitrogen atom to which they are attached form a 4-, 5-, 6- or 7-membered saturated carbocyclic ring, the 4-, 5-, 6- or 7-membered saturated ring optionally further comprising an additional heteroatom group selected from O, N and S(O)ₘ (where m is 0, 1, or 2)), C₃₋₇cycloalkylCᵢ₋₄alkoxy, C₁₋₄alkylthio, and haloC₁₋₄alkylthio;
R³ and R⁴ are independently selected from the group consisting of hydrogen and C₁₋₄alkyl, optionally substituted with one or more groups Y; or R³ and R⁴ together with the nitrogen atom to which they are attached form a saturated or partially unsaturated 4-, 5- 6- or 7-membered carbocyclic ring optionally substituted with a group Y′;
Y is selected from the group consisting of C₁₋₄alkoxy, hydroxy, haloC₁₋₄alkoxy and C₃₋₅cycloalkyl;
Y′ is selected from the group consisting of C₁₋₄alkyl, C₁₋₄alkoxy, halogen, hydroxy, haloC₁₋₄alkoxy, C₃₋₅cycloalkyl and C₅₋₁₀aryl or Y′ forms a —CHᵣ or —CH₂—CH₂— bridge between two atoms on the 4-, 5- or 6-membered carbocyclic ring;
R⁵ and R⁶ are independently C₁₋₄alkyl, optionally substituted with one or more groups X; or R⁵ and R⁶ together with the carbon atom to which they are attached form a saturated 5- or 6-membered carbocyclic ring optionally substituted with one or more groups X′, in the case of R⁵ and R⁶ together with the carbon atom to which they are attached forming a 5-membered saturated carbocyclic ring, that ring may optionally further comprising an additional heteroatom group selected from O, N and S(O)ₘ; where m=0, 1 or 2,
X is selected from the group consisting of halogen, hydroxy, C₁₋₄alkoxy, haloC₁₋₄alkyl, haloC₁₋₄alkoxy and C₅₋₁₀aryl;
X′ is selected from the group consisting of halogen, hydroxy, C₁₋₄alkoxy, haloC₁₋₄alkyl, haloC₁₋₄alkoxy and C₅₋₁₀aryl,
and R¹ is selected from a) and b) wherein
a) is a group selected from: 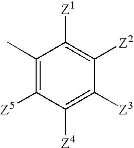
wherein
Z¹ is selected from the group consisting of C₁₋₄alkyl, C₃₋₆cycloalkyl, C₁₋₄alkoxy, C₁₋₄alkylthio, haloC₁₋₄alkyl, phenyl, haloC₁₋₄alkoxy, halophenyl, C₁₋₄alkylsulfoxy, C₁₋₄alkylsulfonyl, bromo and chloro;
Z² is selected from the group consisting of hydrogen, halogen, cyano, C₁₋₄alkyl, phenyl, haloC₁₋₄alkyl, haloC₁₋₄alkoxy, halophenyl, C₁₋₄alkoxyC₁₋₄alkyl and C₃₋₆cycloalkyl;
Z³ is selected from the group consisting of hydrogen, halogen, C₁₋₄alkyl, C₁₋₄alkoxy, C₁₋₄alkylthio, haloC₁₋₄alkyl, haloC₁₋₄alkoxy, and C₃₋₆cycloalkyl;
Z⁴ is selected from the group consisting of hydrogen, halogen, C₁₋₄alkyl, haloC₁₋₄alkyl, C₁₋₄alkoxy, C₁₋₄alkylthio, phenyl, haloC₁₋₄alkoxy, halophenyl, C₁₋₄alkoxyC₁₋₄alkyl and C₃₋₆cycloalkyl;
Z⁵ is selected from the group consisting of hydrogen, fluoro, chloro, bromo, iodo, hydroxy, C₁₋₄alkyl, C₁₋₄alkoxy, C₁₋₄alkylthio, phenyl, haloC₁₋₄alkyl, haloC₁₋₄alkoxy, halophenyl, C₁₋₄alkoxyC₁₋₄alkyl and C₃₋₆cycloalkyl;
whereby if more than one of Z¹ to Z⁵ is methoxy, then only Z¹ and Z⁵ are methoxy;
b) is a group selected from 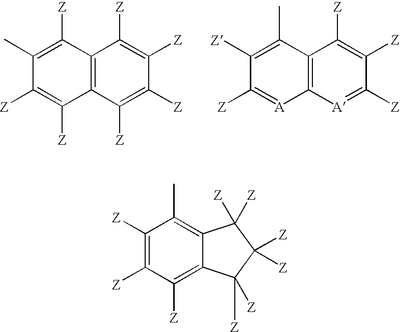
wherein A and A′ are each selected from CZ and Ni, and A and A′ are not both simultaneously N;
Z′ is selected from: hydrogen, halogen, C₃₋₇cycloalkyl, C₁₋₄alkyl, haloC₁₋₄alkyl and C₁₋₄alkoxyC₁₋₄alkyl,
Each Z is independently selected from hydrogen, halogen, C₁₋₄cycloalkyl, C₁₋₄alkyl, haloC₁₋₄alkyl, C₁₋₄alkoxyC₁₋₄alkyl, C₁₋₄alkoxy, haloC₁₋₄alkoxy, C₁₋₄alkylthio, haloC₁₋₄alkylthio, C₁₋₄alkylsulphoxy, C₁₋₄ alkylsulphonyl, C₁₋₄ dialkylamino, furanyl, piperidinyl and cyano;
and at most 2 groups Z (or where appropriate Z and Z′ together) are not hydrogen.

2. A compound as claimed in claim 1 that is a compound of formula (Ia) or a salt or solvate thereof: 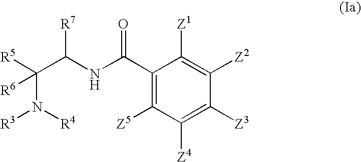
wherein
R⁷ is selected from the group consisting of phenyl substituted with one or more groups R², unsubstituted benzyl, unsubstituted thiophene and unsubstituted C₁₋₄alkyl;
R² is selected from the group consisting of halogen, C₁₋₄alkoxy, C₁₋₄alkyl, haloC₁₋₄alkyl, and haloC₁₋₄alkoxy;
Z¹ is selected from the group consisting of C₁₋₄alkyl, C₁₋₂alkoxy, C₁₋₄alkylthio, haloC₁₋₄alkyl, and chloro;
Z² is selected from the group consisting of hydrogen, halogen, haloC₁₋₄alkyl, and C₁₋₄alkyl;
Z³ is selected from the group consisting of hydrogen, halogen, haloC₁₋₄alkyl and C₁₋₄alkyl;
Z⁴ is selected from the group consisting of hydrogen and halogen;
Z⁵ is selected from the group consisting of bromo, C₁₋₄alkyl, C₁₋₄alkoxy and haloC₁₋₄alkyl;
R³ and R⁴ are independently selected from hydrogen, C₁₋₄alkyl optionally substituted with a group Y, or R³ and R⁴ together with the nitrogen atom to which they are attached form a saturated or partially unsaturated 4-, 5-, 6- or 7-membered carbocyclic ring optionally substituted with a group Y′;
Y is selected from the group consisting of C₁₋₄alkoxy, hydroxy, C₃₋₅cycloalkyl and C₅₋₁₀ aryl,
Y′ is selected from the group consisting of halogen and Cᵢ₋₄alkyl,
R⁵ and R⁶ are independently selected from C₁₋₄alkyl optionally substituted with one or more groups X; or R⁵ and R⁶ together with the carbon atom to which they are attached form a saturated 5- or 6-membered carbocyclic ring and in the case of R⁵ and R⁶ together with the carbon atom to which they are attached forming a 5-membered saturated carbocyclic ring, that ring may optionally further comprise an oxygen heteroatom; and
X is selected from the group consisting of hydroxy and C₁₋₄alkoxy.

3. A compound as claimed in claim 1 or claim 2 which is any of Examples 1 to 26 or a salt or solvate thereof.

4. A compound as claimed in any one of claims 1 to 3 for use in therapy.

5. A compound as claimed in claim 4 for use in the treatment of a disorder mediated by GlyT1.

6. A compound as claimed in claim 5, wherein the disorder is psychosis, including schizophrenia, dementia or attention deficit disorder.

7. A method of treating a mammal, including a human, suffering from or susceptible to a disorder mediated by GlyT1, which comprises administering an effective amount of a compound as claimed in claim 4.

8. A method as claimed in claim 7, wherein the disorder is psychosis, including schizophrenia, dementia or attention deficit disorder.

9. Use of a compound as claimed in any one of claims 1 to 3 in the preparation of a medicament for the treatment of a disorder mediated by GlyT1.

10. Use as claimed in claim 9, wherein the disorder is psychosis, including schizophrenia, dementia or attention deficit disorder.

11. A pharmaceutical composition comprising a compound as claimed in claim 4, and at least one pharmaceutically acceptable carrier, diluent or excipient.

12. A pharmaceutical composition as claimed in claim 11 further comprising one or more other therapeutic agents, selected from antidepressant agents selected from 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 51-1T1B antagonists, 5HT1D antagonists, D1 agonists, M1 agonists, anticonvulsant agents; atypical antipsychotic drugs and cognitive enhancers.

13. A method of preparing a compound as defined in any one of claims 1 to 3, comprising the step of:
reacting a compound of formula (II): 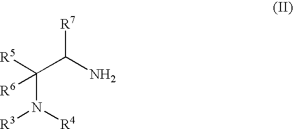
wherein R³, R⁴, R⁶, R⁶ and R⁷ are as defined in formula (I) in any one of claims 1 to 3, with a compound of formula (III): 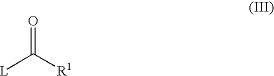
wherein R¹ is as defined in formula (I) in any one of claims 1 to 3 and L represents a suitable leaving group;
and thereafter optionally:
removing any protecting groups and/or
converting a compound of formula (I) into another compound of formula (I) and/or
forming a salt or solvate.

14. A compound of formula (II): 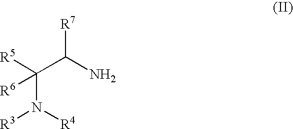
wherein R³, R⁴, R⁵, R⁶ and R⁷ are as defined in formula (I) in any one of claims 1 to 3.
